(19) United States
(12) Patent Application Publication
(10) Pub. No.: US 2019/0218290 A1
(43) Pub. Date: Jul. 18, 2019

(54) CAMEL DERIVED POLYPEPTIDES BINDING HUMAN CD3, IDENTIFIED USING A NOVEL ONE-STEP METHOD

(71) Applicant: ANTI-DOPING LAB QATAR, Doha (QA)

(72) Inventors: Pasquale VITO, Doha (QA); Vidya MOHAMED-ALI, Doha (QA); Mohammed ALSAYRAFI, Doha (QA)

(21) Appl. No.: 16/331,449

(22) PCT Filed: Sep. 7, 2017
(86) PCT No.: PCT/QA2017/050004

§ 371 Date: Mar. 7, 2019

(30) Foreign Application Priority Data
Sep. 7, 2016 (QA) QA/201609/00402

(51) Int. Cl. C07K 16/28; G01N 33/68; A61K 51/10; A61K 49/00; A61K 47/68

(52) U.S. Cl. CPC C07K 16/2809; G01N 33/6872; A61K 51/1042; C07K 2317/14; A61K 47/6849; C07K 2317/22; C07K 2317/569; A61K 49/0058

(57) ABSTRACT

The present invention relates to variable domains of camelid heavy-chain antibodies (VHH domains) directed against the human CD3ε component of the T cell receptor and specifically to methods of use of said polypeptides for delivering prophylactic, therapeutic or diagnostic compounds to human T lymphocytes. It further describes a novel one-step method for the identification of phage displayed camel monoclonal antibodies.

### BACKGROUND OF THE INVENTION

The acquired immune system in vertebrates provides a defence mechanism against foreign intruders, such as extraneous macromolecules or infecting microorganisms. Specifically, the mammalian immune system include two principal classes of lymphocytes: the thymus derived cells (T cells), and the bone marrow derived cells (B cells). Mature T cells emerge from the thymus and circulate between the tissues, lymphatics, and the bloodstream. T cells exhibit immunological specificity and are directly involved in cell-mediated immune responses (such as graft rejection). T cells act against or in response to a variety of foreign structures (antigens). In many instances these foreign antigens are expressed on host cells as a result of infection. However, foreign antigens can also come from the host having been altered by neoplasia or infection. Although T cells do not themselves secrete antibodies, they are usually required for antibody secretion by the second class of lymphocytes, B cells.

There are various subsets of T cells, which are generally defined by antigenic determinants found on their cell surfaces, as well as functional activity and foreign antigen recognition. Some subsets of T cells, such as CD8+ cells, are killer/suppressor cells that play a regulating function in the immune system, while others, such as CD4+ cells, serve to promote inflammatory and humoral responses.

T cell activation is a complex phenomenon that depends on the participation of a variety of cell surface molecules expressed on the responding T cell population. Unlike antibodies that recognize whole or smaller fragments of foreign proteins as antigens, the antigen-specific T cell receptor (TcR) complex interacts with only small peptides of the antigen, which must be presented in the context of major histocompatibility complex (MHC) molecules. These MHC proteins represent another highly polymorphic set of molecules randomly dispersed throughout the species. Thus, activation usually requires the tripartite interaction of the TcR and foreign peptidic antigen bound to the major MHC proteins. The TcR is composed of a disulfide-linked heterodimer, containing two clonally distributed, integral membrane glycoprotein chains, alpha and beta (α and β), or gamma and delta (γ and δ), non-covalently associated with a complex of low molecular weight invariant proteins, commonly designated as CD3 (once referred to as T3). The TcR alpha and beta chains determine antigen specificities. The CD3 structures represent accessory molecules that are the transducing elements of activation signals initiated upon binding of the TcR alpha beta (TcR αβ) to its ligand. It consists of a protein complex and is composed of four distinct chains (FIG. 1). In mammals, the complex contains a CD3γ chain, a CD3δ chain, and two CD3ε chains signalling dimers: CD3δ/ε, CD3γ/ε and/(FIG. 1). The very short length of the extracellular domain (EC) of the zeta chain is preserved through evolution, thought to be needed to correctly assemble the TCR-CD3 complex. These chains associate with the T-cell receptor (TCR) and the ζ-chain (zeta-chain) to generate an activation signal in T lymphocytes. The TCR, ζ-chain, and CD3 molecules together constitute the TCR complex. The CD3γ, CD3δ, and CD3ε chains are highly related cell-surface proteins of the immunoglobulin superfamily containing a single extracellular immunoglobulin domain. Containing aspartate residues, the transmembrane region of the CD3 chains is negatively charged, a characteristic that allows these chains to associate with the positively charged TCR chains. The intracellular tails of the CD3 molecules contain a single conserved motif known as an immunoreceptor tyrosine-based activation motif or ITAM for short, which is essential for the signaling capacity of the TCR. Phosphorylation of the ITAM on CD3 renders the CD3 chain capable of binding an enzyme called ZAP70 (zeta associated protein), a kinase that is important in the signaling cascade of the T cell. Because CD3 is required for T-cell activation, drugs (often monoclonal antibodies) that target it are being investigated as immunosuppressant therapies (e.g., otelixizumab) for type 1 diabetes and other autoimmune diseases.

CD3 is initially expressed in the cytoplasm of pro-thymocytes, the stem cells from which T-cells arise in the thymus. The pro-thymocytes differentiate into common thymocytes, and then into medullary thymocytes, and it is at this latter stage that CD3 antigen begins to migrate to the cell membrane. The antigen is found bound to the membranes of all mature T-cells, and in virtually no other cell type, although it does appear to be present in small amounts in Purkinje cells. This high specificity, combined with the presence of CD3 at all stages of T-cell development, makes it a useful marker for T-cells in tissue sections. The antigen remains present in almost all T-cell lymphomas and leukaemias, and can therefore be used to distinguish them from superficially similar B-cell and myeloid neoplasms.

As part of the immune system, B lymphocytes of vertebrate organisms synthesize antigen-recognizing proteins known as antibodies or immunoglobulins (Ig). According to the clonal selection theory, an antigen activates those B-cells of the host organism that have on their surface immunoglobulins that can recognize and bind the antigen. The binding triggers production of a clone of identical B-cells that secrete soluble antigen-binding immunoglobulins into the bloodstream. Antibodies secreted by B-cells bind to foreign material (antigen), and the complexes antigen-antibody are either recognized or disposed of by macrophages and other effector cells of the immune system or are directly lysed by a family of serum proteins collectively called complement. In this way a small amount of antigen can elicit an amplified and specific immune response that helps to clear the host organism of the source of antigen. Through a stochastic process of genetic rearrangements combined with additional mutation mechanisms, human B-cells have been estimated to produce a “library” (repertoire) of more than a billion (10⁹) different antibodies that differ in the composition of their binding sites. In most vertebrate organisms, including humans and murine species, the basic structural unit of antibodies consists of a glycoprotein (MW ˜150,000 daltons) comprising four polypeptide chains, two identical light chains and two identical heavy chains, which are connected by disulfide bonds, resulting in a Y-shaped molecule. Each light chain has a molecular weight of ˜25,000 daltons and is composed of two domains, one variable domain (VL) and one constant domain (CL). There are two types of light chains, lambda (λ) and kappa (κ). In humans, 60% of the light chains are κ, and 40% are λ, whereas in mice, 95% of the light chains are κ and only 5% are λ. A single antibody molecule contains either κ light chains or λ light chains, but never both. The heavy chains have five different isotypes that divide immunoglobulins into five different functional classes (IgG, IgM, IgA, IgD, IgE), each with different effector properties in the elimination of antigen. Comparison of amino acid sequences between different IgGs shows that the amino-terminal domain of each chain (both light and heavy) is highly variable, whereas the remaining domains have substantially constant sequences. In other words, the light (L) chains of an IgG molecule are built up from one amino-terminal variable domain (VL) and one carboxy-terminal constant domain (CL), and the heavy (H) chains from one amino-terminal variable domain (VH) followed by three constant domains (CH1, CH2, and CH3). The variable domains are not uniformly variable throughout their length. Three small regions of a variable domain, known as hypervariable regions (loops) or complementarity determining regions (CDR1, CDR2, and CDR3) show much more variability than the rest of the domain. These regions, which vary in size and sequence among various immunoglobulins, determine the specificity of the antigen-antibody interaction. The specificity of an antibody of the type is determined by the sequence and size of six hypervariable loops (regions), three in the VL domain and three in the VH domain.

Recently, a new class of antibodies known as heavy chain antibodies (HCA, also referred to as two-chain or two-chain heavy chain antibodies) have been reported in camelids (camels, dromedaries, llamas and alpacas) (Hamers-Casterman et al., Nature, 363, 446-448 (1993); see also U.S. Pat. Nos. 5,759,808; 5,800,988; 5,840,526; and 5,874,541). Compared with conventional four-chain immunoglobulins of IgG-type, which are also produced by camelids, these antibodies lack the light chains and CH1 domains of conventional immunoglobulins (FIG. 2). These heavy-chain antibodies interact with the antigen by the virtue of only one single variable domain, referred to as VHH(s), VHH domain(s) or VHH antibody (ies). Despite the absence of light chain, these homodimeric antibodies exhibit a broad antigen-binding repertoire by enlarging their hypervariable regions. Another feature of camelid HCA is represented by the presence of cysteine pairs in their CDRs. In fact, cysteine pairs mediate the formation of a disulfide bridge and are therefore involved in modulating the surface topology of the antibody combining site (Desmyter et al., Nature Struct. Biol., 3, 803-811 (1996).

Antibody-based therapeutics have significant potential as drugs because they have exquisite specificity to their target and a low inherent toxicity. In addition, the development time can be reduced considerably when compared to the development of new chemical entities. However, the use of antibodies derived from sources such as mouse, sheep, goat, rabbit etc., and humanised derivatives thereof, as a treatment for conditions which require a modulation of immunological responses is problematic for several reasons. Traditional antibodies are not stable at room temperature, and have to be refrigerated for preparation and storage, requiring necessary refrigerated laboratory equipment, storage and transport, which contribute towards time and expense. Refrigeration is sometimes not feasible in developing countries. Furthermore, the manufacture or small-scale production of said antibodies is expensive because the mammalian cellular systems necessary for the expression of intact and active antibodies require high levels of support in terms of time and equipment, and yields are very low. Also the large size of conventional antibodies may restrict tissue penetration, for example, at the site of inflammation. In addition, traditional antibodies have a binding activity which depends upon pH, and hence are unsuitable for use in environments outside the usual physiological pH range. These antibodies are unstable at low or high pH and hence are not suitable for oral administration. They also have a binding activity which depends upon temperature, and hence are unsuitable for use in assays or kits performed at temperatures outside biologically active temperature ranges (e.g. 37±2.0° C.).

Another important drawback of conventional antibodies is that they are complex, large molecules and therefore relatively unstable, and are susceptible to breakdown by proteases. This means that conventional antibody drugs cannot be administered orally, sublingually, topically, nasally, vaginally, rectally or by inhalation because they are not resistant to the low pH at these sites, the action of proteases at these sites and in the blood and/or because of their large size. They have to be administered by injection (intravenously, subcutaneously, etc.) to overcome some of these problems. Administration by injection requires specialist training in order to use a hypodermic syringe or needle correctly and safely. It further requires sterile equipment, a liquid formulation of the therapeutic polypeptide, vial packing of said polypeptide in a sterile and stable form, and in the subjects they need a suitable site for injection. Also, subjects commonly experience physical and psychological stress prior to and upon receiving an injection. Therefore, there is need for a method for the delivery of therapeutic polypeptides which avoids the need for injection, but which would also be more convenient and more comfortable for the subject.

In this context, single domain antibody-based therapeutics have significant potential as drugs because they have exquisite specificity to their target and a low inherent toxicity. In fact, recombinant VHH domains (VHHs) are inherently thermostable (antigen binding of VHH being demonstrated at 90° C.) and exhibit the antigen-binding capacity of the camelid original heavy-chain antibody (Nguyen et al., 2001, Adv. Immunol., 79, 261-96; Muyldermans et al., 2001, Trends in Biochemical Sciences, 26:230-235). VHHs have also been shown to be extremely plastic in that, when they do eventually undergo denaturation, they are often capable of quantitative refolding. Small size (14-17 Kda) and increased plasticity appear to provide VHHs with unique potentialities: for instance, their diffusion into tissues is facilitated by their small size, and several VHHs are capable of inhibiting enzymatic activity by interacting with the active site cavity of enzymes such as alpha-amylase, carbonic anhydrase and hen egg lysozyme (Desmyter et al., 1996, Nature Structural Biology, 3:803-11; Desmyter et al., 2002, Journal of Biological Chemistry, 277:23645-23650; Transue et al., 1998, Proteins, 32:515-22; Lauwereys et al., 1998, Embo J., 17:3512-20). In addition, such antibodies are known to be stable over long periods of time, therefore increasing their shelf-life (Perez et al, Biochemistry, 40, 74, 2001). Furthermore, such heavy chain antibody fragments can be produced ‘en-masse’ in fermentors using cheap expression systems compared to mammalian cell culture fermentation, such as yeast or other microorganisms. Also, it has been demonstrated that camelidae antibodies resist harsh conditions, such as extreme pH, denaturing reagents and high temperatures (Dumoulin et al, Protein Science 11, 500, 2002), so making them suitable for delivery by oral administration. Finally, the recent advances in gene technology have greatly facilitated the genetic manipulation, production, identification and conjugation of recombinant antibody fragments and broadened the potential utility of antibodies as diagnostic and therapeutic agents. Of particular importance to such applications is the possibility to alter the fine specificity of the antibody binding site, to create small stable antigen-binding fragments and to prepare fusion proteins combining antigen-binding domains with proteins having desired therapeutic properties.

### SUMMARY OF THE INVENTION

The present invention relates to the generation and screening of a large size (in the order of 10⁹) phage display library of antibody fragments from an immunized camel. These fragments comprise at least a part of the variable heavy domain (VHH domain) of camelid antibodies. In a preferred embodiment, the fragments consist essentially of the VHH domain of camelid antibodies. From this library, VHH fragments capable of selective binding to the epsilon subunit of the human CD3 complex (CD3E) have been isolated. It is a further aim of the present invention to provide single domain antibodies which may be any of the art, or any future single domain antibodies. Examples include, but are not limited to, heavy chain antibodies, antibodies naturally devoid of light chains, single domain antibodies derived from conventional 4-chain antibodies, engineered antibodies and single domain scaffolds other than those derived from antibodies. According to one aspect of the invention, a single domain antibody as used herein is a naturally occurring single domain antibody known as heavy chain antibody devoid of light chains. For clarity reasons, this variable domain derived from a heavy chain antibody devoid of light chain will be called VHH to distinguish it from the conventional VH of four chain immunoglobulins. Such a VHH molecule can be derived from antibodies raised in Camelidae species, for example in camel, llama, dromedary, alpaca and guanaco.

Accordingly, the present invention provides a camelid VHH directed against human CD3E.

In another embodiment, the present invention is an anti-CD3ε polypeptide comprising at least one anti-CD3ε single domain antibody.

Particularly, the CD3ε is from a warm-blooded animal, more particularly from a mammal, and especially from human origin. For instance, a human CD3ε is available in the GENBANK database under the following accession numbers: gi: 29437136 or gi: 119587764.

A VHH domain refers usually to a variable domain of a camelid (camel, dromedary, llama, and alpaca) heavy-chain antibody (See Nguyen et al., 2001, above-cited; Muyldermans et al., 2001, above-cited).

According to the present invention, a VHH domain comprises an isolated, recombinant or synthetic VHH domain.

As used herein, the term “isolated” refers to a VHH domain which has been separated from a camelid heavy-chain antibody from which it derives.

As used herein, the term “recombinant” refers to the use of genetic engineering methods (cloning, amplification) to produce said VHH domain.

As used herein, the term “synthetic” refers to production by in vitro chemical or enzymatic synthesis.

Preferably, the VHH domain of the invention is from a camel (Camelus dromedarius) heavy-chain antibody.

Preferably, the VHH domain of the invention consists of 100 to 130 amino acid residues. The VHH domain can also be in the form of a dimer, preferably consisting of 245 to 265 amino acid residues.

In a more preferred embodiment, the VHH domain of the invention comprises or consists of the amino acid sequence selected from the group consisting of SEQ ID NOs: 1 to 32 as shown in Table 1.

A VHH domain of the invention is obtainable by the method comprising the steps of:

(a) immunizing a camelid, preferably a Camelus dromedarius, with a CD3ε as defined above,
(b) isolating peripheral lymphocytes of the immunized camelid, obtaining the total RNA and synthesizing the corresponding cDNAs (methods are known in the art)
(c) constructing a library of cDNA fragments encoding VHH domains,
(d) transcribing the VHH domain-encoding cDNAs obtained in step (c) to mRNA using PCR, converting the mRNA to a phage display format, and selecting the VHH domain by phage display screening,
(e) expressing the VHH domain in a vector and, optionally purifying the expressed VHH domain.

The present invention also provides a polypeptide comprising a VHH domain as defined above. When the polypeptide of the present invention comprises at least two VHH domains as defined above, then said VHH domains can be identical or different and can be separated from one another by a spacer, preferably an amino acid spacer.

In order to allow the purification of a polypeptide of the present invention, said polypeptide can contain at its C-terminus an His-tag, such as the amino acid sequence GQHHHHHH (SEQ ID NO: 33).

Therefore, in an embodiment of said polypeptide, it further contains at the C-terminus of its amino acid sequence the amino acid sequence GQHHHHHH (SEQ ID NO: 33). By way of example, the polypeptides of amino acid sequences SEQ ID NO: 34, SEQ ID NO: 35 and SEQ ID NO: 36 consist of the VHH domains of sequences SEQ ID NO: 1, 2, and 3 respectively to which the amino acid sequence GQHHHHHH (SEQ ID NO: 33) has been fused.

The present invention also provides isolated antibodies, preferably camelid heavy-chain antibodies, or fragments thereof, comprising a VHH domain of the invention, wherein said isolated antibodies or fragments thereof bind to a CD3ε as defined above.

As used herein, the terms “antibody fragment” means a portion of a full-length (whole) antibody, e.g., only one heavy chain or the Fab region.

The present invention also provides isolated polynucleotides encoding a VHH domain, a polypeptide, or an antibody or fragment thereof of the present invention. Polynucleotides of the invention may be obtained by the well-known methods of recombinant DNA technology and/or of chemical DNA synthesis.

In a particular embodiment of said polynucleotide, it is a cDNA derived from a gene encoding a VHH domain with no hinge or with a long hinge.

The present invention also provides recombinant expression cassettes comprising a polynucleotide of the invention under the control of a transcriptional promoter allowing the regulation of the transcription of said polynucleotide in a host cell. Said polynucleotide can also be linked to appropriate control sequences allowing the regulation of its translation in a host cell. The present invention also provides recombinant vectors comprising a polynucleotide or an expression cassette of the invention.

The present invention also provides a host cell containing a recombinant expression cassette or a recombinant vector of the invention. The host cell is either a prokaryotic or eukaryotic host cell.

From the above, it would be obvious to those skilled in the art that antibody fragments of the present invention can be used as carriers (vectors) for therapeutic and diagnostic agents to be specifically delivered to the surface of cells expressing CD3E. Such therapeutic or diagnostic agents include hydrophilic molecules, peptides, proteins, pieces of DNA, fluorescently or radioactively labelled compounds, phage particles, liposome formulations, polymer formulations etc. Such agents can be attached to the antibody fragments either directly or indirectly (e.g., via suitable linkers), either by covalent or noncovalent bonds, for example by using complementary pieces of DNA attached to the antibody fragment and the molecule of a therapeutic or diagnostic agent.

The present invention also provides a therapeutic or diagnostic agent comprising a VHH domain, polypeptide or antibody of the present invention, linked, directly or indirectly, covalently or non-covalently to a substance of interest.

In an embodiment of said therapeutic or diagnostic agent, said substance of interest is a therapeutic or diagnostic compound selected from the group consisting of a peptide, an enzyme, a nucleic acid, a virus, a fluorophore, a heavy metal, a chemical entity and a radioisotope.

In another embodiment of said therapeutic or diagnostic agent, the substance of interest is a liposome or a polymeric entity comprising a therapeutic or a diagnostic compound as defined above.

In a preferred embodiment of said diagnostic agent, said diagnostic compound is selected from the group consisting of:

enzymes such as horseradish peroxidase, alkaline phosphatase, glucose-6-phosphatase or □-galactosidase;
fluorophores such as green fluorescent protein (GFP), blue fluorescent dyes excited at wavelengths in the ultraviolet (UV) part of the spectrum (e.g. AMCA (7-amino-4-methylcoumarin-3-acetic acid); Alexa Fluor 350), green fluorescent dyes excited by blue light (e.g. FITC, Cy2, Alexa Fluor 488), red fluorescent dyes excited by green light (e.g. rhodamines, Texas Red, Cy3, Alexa Fluor dyes 546, 564 and 594), or dyes excited with far-red light (e.g. Cy5) to be visualized with electronic detectors (CCD cameras, photomultipliers); heavy metal chelates such as europium, lanthanum or yttrium;
radioisotopes such as [18F]fluorodeoxyglucose, 11C-, 125I-, 131 I-, 3H-, 14C-, 35S, or 99Tc-labelled compounds.

In another preferred embodiment of said therapeutic agent, said therapeutic compound is selected from the group consisting of an anticancer compound, or immunosuppressive, or an anti-inflammatory compound.

The substance of interest as defined above can be directly and covalently or non-covalently linked to the VHH domain, polypeptide or antibody of the present invention either to one of the terminal ends (N or C terminus) of said VHH domain, polypeptide or antibody, or to the side chain of one of the amino acids of said VHH domain, polypeptide or antibody. The substance of interest can also be indirectly and covalently or non-covalently linked to said VHH domain, polypeptide or antibody by a connecting arm (i.e., a cross-linking reagent) either to one of the terminal ends of said VHH domain, polypeptide or antibody, or to a side chain of one of the amino acids of said VHH domain, polypeptide or antibody. Linking methods of a substance of interest to a peptide, in particular an antibody, are known in the art (e.g., Ternynck and Avrameas, 1987, “Techniques immunoenzymatiques” Ed. INSERM, Paris).

Alternatively, if the substance of interest is a peptide, the VHH domain, polypeptide or antibody of the present invention and said substance of interest can be produced by genetic engineering as a fusion polypeptide that includes the VHH domain, polypeptide or antibody of the invention and the suitable peptide. This fusion polypeptide can conveniently be expressed in known suitable host cells.

The VHH domain, the polypeptide, the antibody, the therapeutic or diagnostic agent, or the polynucleotide of the present invention can be administered to a subject (α mammal or a human) by injection, such as intravenous, intraperitoneal, intramuscular or subcutaneous injection.

A diagnostic agent of the present invention can be used in imaging, in diagnosing or monitoring T lymphocytes-involving disorders, such as lymphoproliferative, autoimmune and inflammatory disorders.

The present invention also provides a pharmaceutical composition comprising a therapeutic agent as defined above and a pharmaceutically acceptable carrier.

As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field. Preferred examples of such carriers or diluents include, but are not limited to, water, saline, Ringer's solutions, dextrose solution, and δ% human serum albumin. Liposomes, cationic lipids and non-aqueous vehicles such as fixed oils may also be used. The use of such media and agents for pharmaceutically active substances is well known in the art. Except insofar as any conventional media or agent is incompatible with a therapeutic agent as defined here above, use thereof in the composition of the present invention is contemplated.

As used herein, the term “treatment” includes the administration of the VHH domain, VHH fragment, polypeptide, polynucleotide, therapeutic agent or a pharmaceutical composition as defined above to a patient with the purpose to cure, heal, alleviate, relieve, alter, remedy, ameliorate or improve its health status.

### DETAILED DESCRIPTION OF THE INVENTION

The present invention relates to an anti-CD3epsilon (CD3ε) polypeptide, comprising one or more single domain antibodies which are directed against CD3ε. The invention also relates to nucleic acids capable of encoding said polypeptides.

Another embodiment of the present invention is an anti-CD3ε polypeptide wherein at least one single domain antibody corresponds to a sequence corresponding to any of SEQ ID NOs: 1 to 32 as shown in Table 1. Said sequences are derived from Camelidae heavy chain antibodies (VHHs) which are directed towards CD3ε.

Single domain antibodies are antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any of the art, or any future single domain antibodies. Single domain antibodies may be derived from any species including, but not limited to mouse, human, camel, llama, goat, rabbit, bovine. According to one aspect of the invention, a single domain antibodies as used herein is a naturally occurring single domain antibody known as heavy chain antibody devoid of light chains. Such single domain antibodies are disclosed in WO 94/04678 for example. For clarity reasons, this variable domain derived from a heavy chain antibody naturally devoid of light chain is known herein as a VHH to distinguish it from the conventional VH of four chain immunoglobulins. Such a VHH molecule can be derived from antibodies raised in Camelidae species, for example in camel, dromedary, llama, vicuña, alpaca and guanaco. Other species besides Camelidae may produce heavy chain antibodies naturally devoid of light chain; such VHHs are within the scope of the invention.

VHHs, according to the present invention, and as known to the skilled addressee are heavy chain variable domains derived from immunoglobulins naturally devoid of light chains such as those derived from Camelidae as described in WO 94/04678 (and referred to hereinafter as VHH domains). VHH molecules are about 10× smaller than IgG molecules. They are single polypeptides and very stable, resisting extreme pH and temperature conditions. Moreover, they are resistant to the action of proteases which is not the case for conventional antibodies. Furthermore, in vitro expression of VHHs produces high yield, properly folded functional VHHs. In addition, antibodies generated in Camelids will recognize epitopes other than those recognised by antibodies generated in vitro through the use of antibody libraries or via immunisation of mammals other than Camelids (WO 9749805). As such, anti CD3ε VHH's may interact more efficiently with CD3ε than conventional antibodies. Since VHH's are known to bind into ‘unusual’ epitopes such as cavities or grooves (WO 97/49805), the affinity of such VHH's may be more suitable for therapeutic treatment.

The term “specifically binds”, when used to describe binding of an antibody to a target molecule, refers to binding to a target molecule in a heterogeneous mixture of other polypeptides.

The phrases “substantially lack binding” or “substantially no binding”, as used herein to describe binding of an antibody to a control polypeptide or sample, refers to a level of binding that encompasses non-specific or background binding, but does not include specific binding.

Another embodiment of the present invention is an anti-CD3ε consisting of a sequence corresponding to that of a Camelidae VHH directed towards CD3ε or a closely related family member. The invention also relates to a homologous sequence, a function portion or a functional portion of a homologous sequence of said polypeptide. The invention also relates to nucleic acids capable of encoding said polypeptides.

A single domain antibody of the present invention is directed against CD3ε or a closely related family member.

CD3ε is a principal target according to the invention. According to the invention, as and discussed below, a polypeptide construct may further comprise single domain antibodies directed against other targets such as, for example, serum albumin. A single domain antibody directed against a target means a single domain antibody that is capable of binding to said target with an affinity of better than 1×10⁶M.

Targets may also be fragments of said targets. Thus a target is also a fragment of said target, capable of eliciting an immune response. A target is also a fragment of said target, capable of binding to a single domain antibody raised against the full length target.

A fragment as used herein refers to less than 100% of the sequence (e.g., 99%, 90%, 80%, 70%, 60%, 50%, 40%, 30%, 20%, 10% etc.), but comprising 5, 6, 7, 8, 9, 10, 12, 13, 14, 15, 16, 17, 18, 19, 20, 21, 22, 23, 24, 25 or more amino acids. A fragment is of sufficient length such that the interaction of interest is maintained with affinity of 1×10⁻⁶M or better.

A fragment as used herein also refers to optional insertions, deletions and substitutions of one or more amino acids which do not substantially alter the ability of the target to bind to a single domain antibody raised against the wild-type target. The number of amino acid insertions deletions or substitutions is preferably up to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69 or 70 amino acids.

The present invention further relates to an anti-CD3ε polypeptide, wherein a single domain antibodies is a VHH belonging to a class having human-like sequences.

One such class is characterized in that the VHHs carry an amino acid from the group consisting of glycine, alanine, valine, leucine, isoleucine, proline, phenylalanine, tyrosine, tryptophan, methionine, serine, threonine, asparagine, or glutamine at position 45, such as, for example, L45 and a tryptophan at position 103, according to the Kabat numbering. As such, polypeptides belonging to this class show a high amino acid sequence homology to human VH framework regions and said polypeptides might be administered to a human directly without expectation of an unwanted immune response therefrom, and without the burden of further humanisation.

Another human-like class of Camelidae single domain antibodies has been described in WO 03/035694 and contain the hydrophobic FR2 residues typically found in conventional antibodies of human origin or from other species, but compensating this loss in hydrophilicity by the charged arginine residue on position 103 that substitutes the conserved tryptophan residue present in VH from double-chain antibodies. As such, peptides belonging to these two classes show a high amino acid sequence homology to human VH framework regions and said peptides might be administered to a human directly without expectation of an unwanted immune response therefrom, and without the burden of further humanization. The invention also relates to nucleic acids capable of encoding said polypeptides.

Any of the anti-CD3ε VHHs disclosed herein may be of the traditional class or of a class of human-like Camelidae antibodies. Said antibodies may be directed against whole CD3ε or a fragment thereof, or a fragment of a homologous sequence thereof. These polypeptides include the full length Camelidae antibodies, namely Fc and VHH domains.

Another embodiment of the present invention is a multivalent anti-CD3ε polypeptide as disclosed herein comprising at least two single domain antibodies directed against CD3ε. Such multivalent anti-CD3ε polypeptides have the advantage of unusually high functional affinity for the target, displaying much higher than expected properties compared to their monovalent counterparts.

A multivalent anti-CD3ε polypeptide as used herein refers to a polypeptide comprising two or more anti-CD3ε polypeptides which have been covalently linked. The anti-CD3ε polypeptides may be identical in sequence or may be different in sequence, but are directed against the same target or antigen. Depending on the number of anti-CD3ε polypeptides linked, a multivalent anti-CD3ε polypeptide may be bivalent (2 anti-CD3ε polypeptides), trivalent (3 anti-CD3ε polypeptides), tetravalent (4 anti-CD3ε polypeptides) or have a higher valency molecules. According to one aspect of the present invention, the anti-CD3ε polypeptides are linked to each other directly, without use of a linker. According to another aspect of the present invention, the anti-CD3ε polypeptides are linked to each other via a peptide linker sequence. Such linker sequence may be a naturally occurring sequence or a non-naturally occurring sequence. The linker sequence is expected to be non-immunogenic in the subject to which the anti-CD3ε polypeptides is administered. The linker sequence may provide sufficient flexibility to the multivalent anti-CD3ε polypeptide, at the same time being resistant to proteolytic degradation. A non-limiting example of a linker sequences is one that can be derived from the hinge region of VHHs described in WO 96/34103.

It is an aspect of the invention that a multivalent anti-CD3ε polypeptides disclosed above may be used instead of or as well as the single unit anti-CD3ε polypeptides in the therapies and methods of delivery as mentioned herein.

The single domain antibodies may be joined to form any of the anti-CD3ε polypeptides disclosed herein comprising more than one single domain antibody using methods known in the art or any future method. They may be joined non-covalently (e.g. using streptavidin/biotin combination, antibody/tag combination) or covalently. They may be fused by chemical cross-linking by reacting amino acid residues with an organic derivatising agent such as described by Blattler et al, Biochemistry 24, 1517-1524; EP294703. Alternatively, the single domain antibody may be fused genetically at the DNA level i.e. anti-CD3ε polypeptide formed which encodes the complete polypeptide comprising one or more anti-CD3ε single domain antibodies. A method for producing bivalent or multivalent anti-CD3ε polypeptide is disclosed in PCT patent application WO 96/34103. One way of joining VHH antibodies is via the genetic route by linking a VHH antibody coding sequences either directly or via a peptide linker. For example, the C-terminal end of the VHH antibody may be linked to the N-terminal end of the next single domain antibody.

This linking mode can be extended in order to link additional single domain antibodies for the construction and production of tri-, tetra-, etc. functional constructs.

According to one aspect of the present invention, the single domain antibodies are linked to each other via a peptide linker sequence. Such linker sequence may be a naturally occurring sequence or a non-naturally occurring sequence. The linker sequence is expected to be non-immunogenic in the subject to which the anti-CD3ε polypeptide is administered. The linker sequence may provide sufficient flexibility to the anti-CD3ε polypeptide, at the same time being resistant to proteolytic degradation. A non-limiting example of a linker sequences is one that can be derived from the hinge region of VHHs described in WO 96/34103.

The polypeptide disclosed herein may be made by the skilled artisan according to methods known in the art or any future method. For example, VHHs may be obtained using methods known in the art such as by immunizing a camel and obtaining hybridomas therefrom, or by cloning a library of single domain antibodies using molecular biology techniques known in the art and subsequent selection by ELISA with individual clones of unselected libraries or by using phage display.

According to an aspect of the invention an anti-CD3ε polypeptide may be a homologous sequence of a full-length anti-CD3ε polypeptide. According to another aspect of the invention, an anti-CD3ε polypeptide may be a functional portion of a full-length anti-CD3ε polypeptide. According to an aspect of the invention an anti-CD3ε polypeptide may comprise a sequence of an anti-CD3ε polypeptide.

According to an aspect of the invention a single domain antibody used to form an anti-CD3ε polypeptide may be a complete single domain antibody (e.g. a VHH) or a homologous sequence thereof. According to another aspect of the invention, a single domain antibody used to form an anti-CD3ε polypeptide may be a functional portion of a complete single domain antibody. According to another aspect of the invention, a single domain antibody used to form an anti-CD3ε polypeptide may be a homologous sequence of a complete single domain antibody. According to another aspect of the invention, a single domain antibody used to form an anti-CD3ε polypeptide may be a functional portion of a homologous sequence of a complete single domain antibody.

As used herein, a homologous sequence of the present invention may comprise additions, deletions or substitutions of one or more amino acids, which do not substantially alter the functional characteristics of the polypeptides of the invention. For the anti-CD3ε polypeptides, the number of amino acid deletions or substitutions is preferably up to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69 or 70 amino acids.

A homologous sequence according to the present invention may be a sequence modified by the addition, deletion or substitution of amino acids, said modification not substantially altering the functional characteristics compared with the unmodified polypeptide.

A homologous sequence according to the present invention may be a sequence which exists in other Camelidae species such as, for example, camel, dromedary, liama, vicuña, alpaca and guanaco.

Where homologous sequence indicates sequence identity, it means a sequence which presents a high sequence identity (more than 70%, 75%, 80%, 85%, 90%, 95% or 98% sequence identity) with the parent sequence and is preferably characterised by similar properties of the parent sequence, namely affinity, said identity calculated using known methods. Alternatively, a homologous sequence may also be any amino acid sequence resulting from allowed substitutions at any number of positions of the parent sequence according to the formula below:

Ser substituted by Ser, Thr, Gly, and Asn;
Arg substituted by one of Arg, His, Gin, Lys, and Glu;
Leu substituted by one of Leu, lie, Phe, Tyr, Met, and Val;
Pro substituted by one of Pro, Gly, Ala, and Thr;
Thr substituted by one of Thr, Pro, Ser, Ala, Gly, His, and Gin;
Ala substituted by one of Ala, Gly, Thr, and Pro;
Val substituted by one of Val, Met, Tyr, Phe, lie, and Leu;
Gly substituted by one of Gly, Ala, Thr, Pro, and Ser;
lie substituted by one of lie, Met, Tyr, Phe, Val, and Leu;
Phe substituted by one of Phe, Trp, Met, Tyr, lie, Val, and Leu;
Tyr substituted by one of Tyr, Trp, Met, Phe, lie, Val, and Leu;
His substituted by one of His, Glu, Lys, Gin, Thr, and Arg;
Gin substituted by one of Gin, Glu, Lys, Asn, His, Thr, and Arg;
Asn substituted by one of Asn, Glu, Asp, Gin, and Ser;
Lys substituted by one of Lys, Glu, Gin, His, and Arg;
Asp substituted by one of Asp, Glu, and Asn;
Glu substituted by one of Glu, Asp, Lys, Asn, Gin, His, and Arg;
Met substituted by one of Met, Phe, lie, Val, Leu, and Tyr.

A homologous nucleotide sequence according to the present invention may refer to nucleotide sequences of more than 50, 100, 200, 300, 400, 500, 600, 800 or 1000 nucleotides able to hybridize to the reverse-complement of the nucleotide sequence capable of encoding the patent sequence, under stringent hybridization conditions (such as the ones described by Sambrook et al., Molecular Cloning, Laboratory Manuel, Cold Spring, Harbor Laboratory press, New York. As used herein, a functional portion refers to a sequence of a single domain antibody that is of sufficient size such that the interaction of interest is maintained with affinity of 1×10⁻⁶ M or better.

Alternatively, a functional portion comprises a partial deletion of the complete amino acid sequence and which still maintains the binding site(s) and protein domain(s) necessary for the binding of and interaction with CD3ε.

As used herein, a functional portion as it refers to the polypeptide sequence an anti-CD3ε polypeptide refers to less than 100% of the sequence (e.g., 99%, 90%, 80%, 70%, 60% 50% etc.), but comprising 5 or more amino acids or 15 or more nucleotides.

A portion as it refers to the polypeptide of an anti-CD3ε polypeptide, refers to less than 100% of the sequence (e.g., 99%, 90%, 80%, 70%, 60% 50% etc.), but comprising 5 or more amino acids or 15 or more nucleotides.

One embodiment of the present invention relates to a method for preparing modified polypeptides based upon camel antibodies by determining the amino acid residues of the antibody variable domain (VHH) which may be modified without diminishing the native affinity of the domain for antigen and while reducing its immunogenicity with respect to a heterologous species; the use of VHHs having modifications at the identified residues which are useful for administration to heterologous species; and to the VHH so modified. More specifically, the invention relates to the preparation of modified VHHs, which are modified for administration to humans, the resulting VHH themselves, and the use of such “humanized” VHHs in the treatment of diseases in humans. By humanized is meant mutated so that immunogenicity upon administration in human patients is minor or non-existent. Humanizing a polypeptide, according to the present invention, comprises a step of replacing one or more of the Camelidae amino acids by their human counterpart as found in the human consensus sequence, without that polypeptide losing its typical character, i.e. the humanisation does not significantly affect the antigen binding capacity of the resulting polypeptide. Such methods are known by the skilled addressee. Humanization of Camelidae single domain antibodies requires the introduction and mutagenesis of a limited amount of amino acids in a single polypeptide chain. This is in contrast to humanization of scFv, Fab′, (Fab′)2 and IgG, which requires the introduction of amino acid changes in two chains, the light and the heavy chain and the preservation of the assembly of both chains.

One embodiment of the present invention is an anti-CD3ε polypeptide as disclosed herein, or a nucleic acid capable of encoding said polypeptide for use in treating, preventing and/or alleviating the symptoms of disorders relating to autoimmune, immunoproliferative and inflammatory diseases.

Another embodiment of the present invention is a use of an anti-CD3ε VHH as disclosed herein, or a nucleic acid capable of encoding said polypeptide for the preparation of a medicament for treating a disorder relating to autoimmune, immunoproliferative and inflammatory diseases. Autoimmune diseases include, for example, Acquired Immunodeficiency Syndrome (AIDS, which is a viral disease with an autoimmune component), alopecia areata, ankylosing spondylitis, antiphospholipid syndrome, autoimmune Addison's disease, autoimmune hemolytic anemia, autoimmune hepatitis, autoimmune inner ear disease (AIED), autoimmune lymphoproliferative syndrome (ALPS), autoimmune thrombocytopenic purpura (ATP), Behcet's disease, cardiomyopathy, celiac sprue-dermatitis hepetiformis; chronic fatigue immune dysfunction syndrome (CFIDS), chronic inflammatory demyelinating polyneuropathy (CIPD), cicatricial pemphigold, cold agglutinin disease, crest syndrome, Crohn's disease, Degos' disease, dermatomyositis-juvenile, discoid lupus, essential mixed cryoglobulinemia, fibromyalgia-fibromyositis, Graves' disease, Guillain-Barre syndrome, Hashimoto's thyroiditis, idiopathic pulmonary fibrosis, idiopathic thrombocytopenia purpura (ITP), IgA nephropathy, insulin-dependent diabetes mellitus, juvenile chronic arthritis (Still's disease), juvenile rheumatoid arthritis, Meniere's disease, mixed connective tissue disease, multiple sclerosis, myasthenia gravis, pemacious anemia, polyarteritis nodosa, polychondritis, polyglandular syndromes, polymyalgia rheumatica, polymyositis and dermatomyositis, primary agammaglobulinemia, primary biliary cirrhosis, psoriasis, psoriatic arthritis, Raynaud's phenomena, Reiter's syndrome, rheumatic fever, rheumatoid arthritis, sarcoidosis, scleroderma (progressive systemic sclerosis (PSS), also known as systemic sclerosis (SS)), Sjögren's syndrome, stiff-man syndrome, systemic lupus erythematosus, Takayasu arteritis, temporal arteritis/giant cell arteritis, ulcerative colitis, uveitis, vitiligo and Wegener's granulomatosis.

Inflammatory disorders, include, for example, chronic and acute inflammatory disorders. Examples of inflammatory disorders include Alzheimer's disease, asthma, atopic allergy, allergy, atherosclerosis, bronchial asthma, eczema, glomerulonephritis, graft vs. host disease, hemolytic anemias, osteoarthritis, sepsis, stroke, transplantation of tissue and organs, vasculitis, diabetic retinopathy and ventilator induced lung injury.

The present invention provides a therapeutic composition comprising an anti-CD3ε VHH either alone or in combination with a therapeutic agents.

Polypeptides and nucleic acids according to the present invention may be administered to a subject by conventional routes, such as intravenously. However, a special property of the anti-CD3ε polypeptides of the invention is that they are sufficiently small to penetrate barriers such as tissue membranes and/or tumors and act locally and act locally thereon, and they are sufficiently stable to withstand extreme environments such as in the stomach.

Therefore, another aspect of the present invention relates to the delivery of anti-CD3ε polypeptides.

A subject according to the invention can be any mammal susceptible to treatment by therapeutic polypeptides.

Oral delivery of anti-CD3ε polypeptides of the invention results in the provision of such molecules in an active form at local sites that are affected by the disorder. Genetically modified microorganisms such as Micrococcus lactis are able to secrete antibody fragments. Such modified microorganisms can be used as vehicles for local production and delivery of antibody fragments in the intestine.

Another aspect of the invention involves delivering anti-CD3ε polypeptides by using surface expression on or secretion from non-invasive bacteria, such as Gram-positive host organisms like Lactococcus spec. using a vector such as described in WO00/23471.

An aspect of the invention is a method for delivering a T lymphocytes modulator to the bloodstream of a subject without the compound being inactivated, by orally administering to a subject an anti-CD3ε polypeptide as disclosed herein.

Examples of disorders are any that cause inflammation, including but not limited to rheumatoid arthritis and psoriasis. In a non-limiting example, a formulation according to the invention comprises an anti-CD3ε polypeptide as disclosed herein comprising one or more single domain antibodies directed against CD3ε, in the form of a gel, cream, suppository, film, or in the form of a sponge or as a vaginal ring that slowly releases the active ingredient over time (such formulations are described in EP 707473, EP 684814, U.S. Pat. No. 5,629,001).

An aspect of the invention is a method for treating, preventing and/or alleviating the symptoms of disorders susceptible to CD3ε modulators delivered to the vaginal and/or rectal tract, by vaginally and/or rectally administering to a subject an anti-CD3ε polypeptide as disclosed herein.

Another embodiment of the present invention is a use of an anti-CD3ε polypeptide as disclosed herein for the preparation of a medicament for treating, preventing and/or alleviating the symptoms of disorders susceptible to modulation by a CD3ε binding fragment delivered to the vaginal and/or rectal tract.

An aspect of the invention is a method for delivering a CD3ε modulator to the vaginal and/or rectal tract without being said modulator being inactivated, by administering to the vaginal and/or rectal tract of a subject an anti-CD3ε polypeptide as disclosed herein.

An aspect of the invention is a method for delivering a CD3ε modulator to the bloodstream of a subject without said modulator being inactivated, by administering to the vaginal and/or rectal tract of a subject an anti-CD3ε polypeptide as disclosed herein.

Another embodiment of the present invention is an anti-CD3ε polypeptide as disclosed herein, for use in treating, preventing and/or alleviating the symptoms of disorders susceptible to CD3ε modulators delivered to the nose, upper respiratory tract and/or lung.

In a non-limiting example, a formulation according to the invention, comprises an anti-CD3ε polypeptide as disclosed herein directed against CD3ε in the form of a nasal spray (e.g. an aerosol) or inhaler. Since the anti-CD3ε polypeptide is small, it can reach its target much more effectively than therapeutic IgG molecules.

An aspect of the invention is a method for treating, preventing and/or alleviating the symptoms of disorders susceptible to CD3ε modulators delivered to the upper respiratory tract and lung, by administering to a subject an anti-CD3ε polypeptide as disclosed herein, by inhalation through the mouth or nose.

Another embodiment of the present invention is a use of a CD3ε polypeptide as disclosed herein for the preparation of a medicament for treating, preventing and/or alleviating the symptoms of disorders susceptible to modulation by a CD3ε binding fragment delivered to the nose, upper respiratory tract and/or lung, without said polypeptide being inactivated.

An aspect of the invention is a method for delivering a CD3ε modulator to the nose, upper respiratory tract and lung without inactivation, by administering to the nose, upper respiratory tract and/or lung of a subject an anti-CD3ε polypeptide as disclosed herein.

An aspect of the invention is a method for delivering a CD3ε modulator to the bloodstream of a subject without inactivation by administering to the nose, upper respiratory tract and/or lung of a subject an anti-CD3ε polypeptide as disclosed herein.

One embodiment of the present invention is an anti-CD3ε polypeptide as disclosed herein for use in treating, preventing and/or alleviating the symptoms of disorders susceptible to CD3ε modulators delivered to the intestinal mucosa, wherein said disorder increases the permeability of the intestinal mucosa. Because of their small size, an anti-CD3ε polypeptide as disclosed herein can pass through the intestinal mucosa and reach the bloodstream more efficiently in subjects suffering from disorders which cause an increase in the permeability of the intestinal mucosa, for example Crohn's disease.

An aspect of the invention is a method for treating, preventing and/or alleviating the symptoms of disorders susceptible to CD3ε modulators delivered to the intestinal mucosa, wherein said disorder increases the permeability of the intestinal mucosa, by orally administering to a subject an anti-CD3ε polypeptide as disclosed herein.

This process can be even further enhanced by an additional aspect of the present invention—the use of active transport carriers. In this aspect of the invention, VHH is fused to a carrier that enhances the transfer through the intestinal wall into the bloodstream. In a non-limiting example, this “carrier” is a second VHH which is fused to the therapeutic VHH. Such fusion constructs are made using methods known in the art. The “carrier” VHH binds specifically to a receptor on the intestinal wall which induces an active transfer through the wall.

Another embodiment of the present invention is a use of an anti-CD3ε polypeptide as disclosed herein for the preparation of a medicament for treating, preventing and/or alleviating the symptoms of disorders susceptible CD3ε modulators delivered to the intestinal mucosa, wherein said disorder increases the permeability of the intestinal mucosa.

An aspect of the invention is a method for delivering an CD3ε modulator to the intestinal mucosa without being inactivated, by administering orally to a subject an anti-CD3ε polypeptide comprising one or more single domain antibodies directed against CD3ε.

An aspect of the invention is a method for delivering a CD3ε modulator to the bloodstream of a subject without being inactivated, by administering orally to a subject an anti-CD3ε polypeptide comprising one or more single domain antibodies directed against CD3ε.

This process can be even further enhanced by an additional aspect of the present invention—the use of active transport carriers. In this aspect of the invention, a CD3ε polypeptide as described herein is fused to a carrier that enhances the transfer through the intestinal wall into the bloodstream. In a non-limiting example, this “carrier” is a VHH which is fused to said polypeptide. Such fusion constructs made using methods known in the art. The “carrier” VHH binds specifically to a receptor on the intestinal wall which induces an active transfer through the wall.

One embodiment of the present invention is an anti-CD3ε polypeptide as disclosed herein for use in treating, preventing and/or alleviating the symptoms of disorders susceptible to CD3ε modulator that is able to pass through the tissues beneath the tongue effectively. A formulation of said anti-CD3Epolypeptide as disclosed herein, for example, a tablet, spray, drop is placed under the tongue and adsorbed through the mucus membranes into the capillary network under the tongue.

An aspect of the invention is a method for treating, preventing and/or alleviating the symptoms of disorders susceptible to modulation by a therapeutic compound that is able to pass through the tissues beneath the tongue effectively, by sublingually administering to a subject an anti-CD3ε polypeptide as disclosed herein.

Another embodiment of the present invention is a use of an anti-CD3ε polypeptide as disclosed herein for the preparation of a medicament for treating, preventing and/or alleviating the symptoms of disorders susceptible to a CD3ε modulator that is able to pass through the tissues beneath the tongue.

An aspect of the invention is a method for delivering a CD3ε modulator to the tissues beneath the tongue without being inactivated, by administering sublingually to a subject an anti-CD3ε polypeptide as disclosed herein.

An aspect of the invention is a method for delivering a CD3ε modulator to the bloodstream of a subject without being inactivated, by administering orally to a subject an anti-CD3ε polypeptide as disclosed herein.

One embodiment of the present invention is an anti-CD3ε polypeptide as disclosed herein for use in treating, preventing and/or alleviating the symptoms of disorders susceptible to a CD3ε modulator that is able to pass through the skin effectively.

Examples of disorders are cancers and any that cause inflammation, including but not limited to rheumatoid arthritis and psoriasis. A formulation of said an anti-CD3ε polypeptide, for example, a cream, film, spray, drop, patch, is placed on the skin and passes through.

An aspect of the invention is a method for treating, preventing and/or alleviating the symptoms of disorders susceptible to a CD3ε modulator that is able to pass through the skin effectively, by topically administering to a subject an anti-CD3ε polypeptide as disclosed herein.

Another embodiment of the present invention is a use of an anti-CD3ε polypeptide as disclosed herein for the preparation of a medicament for treating, preventing and/or alleviating the symptoms of disorders susceptible to modulation by a CD3ε modulator that is able pass through the skin effectively.

An aspect of the invention is a method for delivering a CD3ε modulator to the skin without being inactivated, by administering topically to a subject an anti-CD3ε polypeptide as disclosed herein.

An aspect of the invention is a method for delivering a CD3ε modulator to the bloodstream of a subject, by administering topically to a subject an anti-CD3ε polypeptide as disclosed herein. In another embodiment of the present invention, an anti-CD3ε polypeptide further comprises a carrier single domain antibody (e.g. VHH) which acts as an active transport carrier for transport said anti-CD3ε polypeptide, the lung lumen to the blood.

Examples of disorders are any due to autoimmunity and/or inflammation. The anti-CD3ε polypeptide further comprising a carrier binds specifically to a receptor present on the mucosal surface (bronchial epithelial cells) resulting in the active transport of the polypeptide from the lung lumen to the blood. The carrier single domain antibody may be fused to the anti-CD3ε polypeptide. Such fusion constructs made using methods known in the art and are describe herein. The “carrier” single domain antibody binds specifically to a receptor on the mucosal surface which induces an active transfer through the surface.

Another aspect of the present invention is a method to determine which single domain antibodies (e.g. VHHs) are actively transported into the bloodstream upon nasal administration. Similarly, a naïve or immune VHH phage library can be administered nasally, and after different time points after administration, blood or organs can be isolated to rescue phages that have been actively transported to the bloodstream. A non-limiting example of a receptor for active transport from the lung lumen to the bloodstream is the Fc receptor N (FcRn). One aspect of the invention includes the VHH molecules identified by the method. Such VHH can then be used as a carrier VHH for the delivery of a therapeutic VHH to the corresponding target in the bloodstream upon nasal administration.

A cell that is useful according to the invention is preferably selected from the group consisting of bacterial cells such as, for example, E. coli, yeast cells such as, for example, S. cerevisiae, P. pastoris, insect cells or mammalian cells.

A cell that is useful according to the invention can be any cell into which a nucleic acid sequence encoding a polypeptide comprising an anti-CD3ε of the invention, an homologous sequence thereof, a functional portion thereof, a functional portion of an homologous sequence thereof or a mutant variant thereof according to the invention can be introduced such that the polypeptide is expressed at natural levels or above natural levels, as defined herein. Preferably a polypeptide of the invention that is expressed in a cell exhibits normal or near normal pharmacology, as defined herein. Most preferably a polypeptide of the invention that is expressed in a cell comprises the nucleotide sequence capable of encoding any one of the amino acid sequences presented in Table 1 or capable of encoding an amino acid sequence that is at least 70% identical to the amino acid sequence presented in Table 1.

According to a preferred embodiment of the present invention, a cell is selected from the group consisting of COS7-cells, a CHO cell, a LM (TK-) cell, a NIH-3T3 cell, HEK-293 cell, K-562 cell or a 1321 N1 astrocytoma cell but also other transfectable cell lines.

In general, “therapeutically effective amount”, “therapeutically effective dose” and “effective amount” means the amount needed to achieve the desired result or results. One of ordinary skill in the art will recognize that the potency and, therefore, an “effective amount” can vary for the various compounds that modulate the CD3ε binding used in the invention. One skilled in the art can readily assess the potency of the compound.

As used herein, the term “compound” refers to an anti-CD3ε polypeptide of the present invention, or a nucleic acid capable of encoding said polypeptide or an agent identified according to the screening method described herein or said polypeptide comprising one or more derivatized amino acids.

By “pharmaceutically acceptable” is meant a material that is not biologically or otherwise undesirable, i.e., the material may be administered to an individual along with the compound without causing any undesirable biological effects or interacting in a deleterious manner with any of the other components of the pharmaceutical composition in which it is contained.

Polypeptides of a human-like class of VHH's as disclosed herein is useful for treating or preventing conditions in a subject and comprises administering a pharmaceutically effective amount of a compound or composition.

Polypeptides of the present invention are useful for treating or preventing conditions relating to autoimmune, immunoproliferative and inflammatory diseases in a subject and comprises administering a pharmaceutically effective amount of a compound or composition that binds CD3ε.

The anti-CD3ε polypeptides as disclosed here in are useful for treating or preventing conditions relating to autoimmune, immunoproliferative and inflammatory diseases in a subject and comprises administering a pharmaceutically effective amount of a compound in combination with another.

The present invention is not limited to the administration of formulations comprising a single compound of the invention. It is within the scope of the invention to provide combination treatments wherein a formulation is administered to a patient in need thereof that comprises more than one compound of the invention.

A compound useful in the present invention can be formulated as pharmaceutical compositions and administered to a mammalian host, such as a human patient or a domestic animal in a variety of forms adapted to the chosen route of administration, i.e., orally or parenterally, intranassally by inhalation, intravenous, intramuscular, topical or subcutaneous routes.

A compound of the present invention can also be administered using gene therapy methods of delivery. See, e.g., U.S. Pat. No. 5,399,346, which is incorporated by reference in its entirety. Using a gene therapy method of delivery, primary cells transfected with the gene for the compound of the present invention can additionally be transfected with tissue specific promoters to target specific organs, tissue, grafts, tumors, or cells and can additionally be transfected with signal and stabilization sequences for subcellularly localized expression.

Thus, the present compound may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form must be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compound may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, hydroxyalkyls or glycols or water-alcohol/glycol blends, in which the present compound can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compound to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the compound can be determined by comparing their in vitro activity, and in vivo activity in animal models. Methods for the extrapolation of effective dosages in mice, and other animals, to humans are known to the art; for example, see U.S. Pat. No. 4,938,949.

Generally, the concentration of the compound(s) in a liquid composition, such as a lotion, will be from about 0.1-25 wt-%, preferably from about 0.5-10 wt-%. The concentration in a semi-solid or solid composition such as a gel or a powder will be about 0.1-5.0 wt-%, preferably about 0.5-2.5 wt-%.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician. Also the dosage of the compound varies depending on the target cell, tumor, tissue, graft, or organ.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

An administration regimen could include long-term, daily treatment. By “long-term” is meant at least two weeks and preferably, several weeks, months, or years of duration. Necessary modifications in this dosage range may be determined by one of ordinary skill in the art using only routine experimentation given the teachings herein. See Remington's Pharmaceutical Sciences (Martin, E. W., ed. 4), Mack Publishing Co., Easton, Pa. The dosage can also be adjusted by the individual physician in the event of any complication.

Another aspect of the invention is a kit containing an anti-CD3ε polypeptide and a least another polypeptide for simultaneous, separate or sequential administration to a subject. It is an aspect of the invention that the kit may be used according to the invention. It is an aspect of the invention that the kit may be used to treat immunological diseases.

By simultaneous administration means the polypeptides are administered to a subject at the same time. For example, as a mixture of the polypeptides or a composition comprising said polypeptides. Examples include, but are not limited to a solution administered intravenously, a tablet, liquid, topical cream, etc., wherein each preparation comprises the polypeptides of interest. By separate administration means the polypeptides are administered to a subject at the same time or substantially the same time. The polypeptides are present in the kit as separate, unmixed preparations. For example, the different polypeptides may be present in the kit as individual tablets. The tablets may be administered to the subject by swallowing both tablets at the same time, or one tablet directly following the other. By sequential administration means the polypeptides are administered to a subject sequentially. The polypeptides are present in the kit as separate, unmixed preparations. There is a time interval between doses. For example, one polypeptide might be administered up to 336, 312, 288, 264, 240, 216, 192, 168, 144, 120, 96, 72, 48, 24, 20, 16, 12, 8, 4, 2, 1, or 0.5 hours after the other component. In sequential administration, one polypeptide may be administered once, or any number of times and in various doses before and/or after administration of another polypeptide. Sequential administration may be combined with simultaneous or sequential administration.

### Methods

The invention is illustrated by the following non-limiting example.

### Example 1: Induction of a Humoral Immune Response in Camel

250 μl of recombinant human CD3ε (1 mg/ml) (TABLE II) was mixed with 250 μl of Freund complete adjuvant for the first immunization, and with 250 μl of Freund incomplete adjuvant for the following immunizations. One young adult male camel (Camelus dromedarius) was immunized at days 0, 21, 35, 49 and 70.

### Example 2: Evaluation of Immune Response

At day 0, 22, 36, 50 and 71, 10 ml of pre-immune/immune blood was collected and serum was used to evaluate the induction of the immune responses in the immunized camel. For this, recombinant human CD3ε and the irrelevant recombinant human CD80, both at 1 μg/ml, were immobilized overnight at 4° C. in a 96 well Maxisorp plate (Nunc). Wells were blocked with a BSA solution (5% in PBS). After addition of serum dilutions, specifically bound immunoglobulins were detected using a HRP-conjugated goat anti-camel antibody, showing that a significant antibody dependent immune response against CD3ε was induced (FIG. 3).

### Example 3: Library Construction

The blood of the immunized animal was collected in Pax tubes (Qiagen) and total RNA from peripheral blood cells extracted according to the manufacturer's instruction. RNA was retro-transcribed in cDNA using the High-Capacity cDNA Reverse Transcription Kit (Thermo Fisher). Over the years, several PCR strategies have been developed to amplify VHH gene fragments from lymphocyte cDNA. We have used a two-step nested PCR approach. One pair of primers, CALL001 (SEQ. ID 37) and CALL002 (SEQ. ID 38) has been designed for the first PCR by using the cDNA as the template. The CALL002 primer anneals in a region of the second constant heavy-chain domain (CH2) that is conserved among all IgG isotypes of all camelids, whereas the CALL001 primer anneals in a well-conserved region of the leader signal sequence of all V elements of family III (by far the most abundant V family in camelids). The amplified product of approximately 600 bp was subjected to a second round of using the primers VHH-Back (SEQ. ID 39) and VHH-For (SEQ. ID 40) to amplify the VHH repertoire (FIG. 4). Following gel electrophoresis, the DNA fragment of approximately 400 base pairs were purified from gel and ligated into the corresponding restriction sites of a phagemid plasmid to obtain a library of cloned VHHs as gpIII fusion protein after electroporation of Escherichia coli TG1. The size of the library was 1.4×10⁹ cfu, and all clones contained insert of the correct size.

### Example 4: Rescue of the Library and Phage Preparation

The library was grown at 37° C. in 10 ml 2×TY medium containing 2% glucose, and 100 μg/ml ampicillin, until the OD600 nm reached 0.5. M13K07 phages (10¹²) were added and the mixture was incubated at 37° C. for 2×30 minutes, first without shaking, then with shaking at 100 rpm. Cells were centrifuged for 10 minutes at 4500 rpm at room temperature. The bacterial pellet was resuspended in 50 ml of 2×TY medium containing 100 μg/ml ampicillin and 25 μg/ml kanamycin, and incubated overnight at 37° C. with vigorously shaking at 250 rpm. The overnight cultures were centrifuged for 15 minutes at 10000 rpm at 4° C. Phages were PEG precipitated (20% poly-ethylene-glycol and 1.5 M NaCl) and centrifuged for 30 minutes at 10000 rpm. The pellet was resuspended in 20 ml PBS. Phages were again PEG precipitated and centrifuged for 30 minutes at 20000 rpm and 4° C. The pellet was dissolved in 5 ml PBS-1% casein. Phages were titrated by infection of TG1 cells at OD600 nm=0.5 and plating on LB agar plates containing 100 μg/ml ampicillin and 2% glucose. The number of transformants indicates the number of phages (=pfu). The phages were stored at −80° C. with 15% glycerol.

### Example 5: Selection Strategy to Identify CD3ε Specific VHHs

Libraries were rescued by growing the bacteria to logarithmic phase (OD600=0.5), followed by infection with helper phage to obtain recombinant phages expressing the repertoire of cloned VHHs on tip of the phage as gpIII fusion protein (as described in Library Construction). When selecting for CD3ε specific antibodies, a novel, original selection strategy was followed (FIG. 5). The first step was based on the fact that the human recombinant CD3ε used for immunization, and containing a hexa-histidine tag (SEQ ID NO: 41), can be bound by affinity chromatography on nickel-agarose beads. The phage library was precleared on nickel-agarose beads for one hour at room temperature in 1 ml of PBS/0.05% Tween-20/5% nonfat dry milk and then probed with agarose-bound CD3ε for 30 minutes at room temperature. Unbound phages were removed by 10 washes with PBS/0.05% Tween-20. Bound phages were subsequently eluted along with CD3ε with 0.5M imidazole. Logarithmically grown E. coli TG1 cells were infected with the eluted phages and grown overnight at 37° C. on selective medium 2×TY and 2% glucose. Only one round of selection was sufficient to enrich for CD3ε specific recombinant phages (FIG. 6).

### Example 6: Expression and Purification of VHH

Free VHH domains were expressed in and purified from E. coli SS320 periplasm by nickel affinity chromatography. In SS320, VHH domains are expressed as free soluble fragments due to lack of amber suppression at the VHH-gene IHp junction. 32 selected colonies were individually used to start an overnight culture in LB containing 2% glucose and 100 μg/ml ampicillin. This overnight culture was diluted 100-fold in 300 ml TB medium containing 100 μg/ml ampicillin, and incubated at 37° C. until OD600 nm=0.5. 1 mM IPTG was added and the culture was incubated for 3 more hours at 37° C. or overnight at 28° C. Cultures were centrifuged for 20 minutes at 10000 rpm at 4° C. The pellet was frozen overnight or for 1 hour at −20° C. Next, the pellet was thawed at room temperature for 40 minutes, re-suspended in 20 ml PBS and shaken on ice for 1 hour. Periplasmic fraction was isolated by centrifugation for 20 minutes at 4° C. at 20000 rpm. The supernatant containing the VHH was loaded on Ni-NTA and purified to homogeneity.

### Example 7: Sequencing of the VHH of the Invention

The cDNA encoding for the selected clones were sequenced using a Big Die Terminator Cycle Sequencing Ready Reaction Kit (Applied Biosystems) in an ABI-Prism 377 DNA automatic sequencer (Perkin Elmer, Applied Biosystems) (FIG. 7).

### Example 8: ELISA: Binding to CD3e

A microtiter plate was coated with 1 μg/ml CD3ε, overnight at 4° C. Plates were blocked for two hours at room temperature with 300 μl 1% BSA in PBS. The plates were washed three times with PBS-Tween. Dilution series of 4 selected purified VHH and 2 purified control VHH randomly picked from a preimmune library were incubated for 2 hours at RT. Plates were washed six times with PBS-Tween, after which binding of VHH was detected by incubation with goat-HRP conjugate anti-HA 1/1000 in PBS for 1 hour at RT. Staining was performed with the substrate ABTS/H202 and the signals were measured after 30 minutes at 405 nm (FIG. 8).

### Example 9: Pull Down Assay

Bacterial lysates containing 4 CD3ε-specific VHH or 2 VHH randomly picked from a preimmune library were probed with Agarose-bound recombinant CD3ε in Tris-buffered saline containing 0.1% (v/v) tween 20 for 1 hour. Lysate were then removed, and the beads were washed 15 times with TBST. Proteins bound to the beads were eluted in SDS-PAGE sample buffer containing 1% (v/v) β-mercaptoethanol and 100 mM imidazole and preheated to 95° C. Samples were subjected to SDS-PAGE and proteins were transferred to a PVDF membrane that was then probed with the anti-HA and a secondary mouse-anti-rabbit IgG-HRP conjugate. The blot was developed with SuperSignal West™ substrate (Pierce) and exposed to film (FIG. 9).

### Example 10: Western Blot

Cell lysates were prepared from Jurkat cells in lysis buffer (150 mM NaCl, 20 mM Hepes, pH 7.4, 1% Triton X-100, 10% glycerol and a mixture of protease inhibitors). Proteins were separated by SDS-PAGE, transferred onto nitrocellulose membrane and incubated with 4 CD3ε-specific VHH or 2 VHH randomly picked from a preimmune library primary antibodies followed by anti-HA and horseradish peroxidase-conjugated secondary antibodies (Amersham Biosciences). The blot was developed with SuperSignal West™ substrate (Pierce) and exposed to film (FIG. 10).

All biochemical and molecular biology reagents were chemical grade purchased from various companies. Unless stated otherwise, the bacterial media were prepared as described (Sambrook et al., Molecular cloning: A Laboratory Manual (2nd Ed.). Cold Spring Harbor Laboratory, Cold Spring Harbor, N.Y. (1989)). Phosphate-buffered saline (PBS) was prepared as described (Sambrook et al., supra). Induction medium was the same as Terrific Broth except that it contained no salts. Agarose top was prepared by combining the following reagents in a total volume of 1 liter: 10 g bacto-tryptone, 5 g yeast extract, 10 g NaCl, 1 g MgCI2.6H2O, and 7 g agarose. The mixture was autoclaved and stored solid at room temperature. The oligonucleotides were synthesized using the Applied Biosystems 394 DNA/RNA synthesizer. DNA sequencing was performed by the dideoxy method (Sanger et al., Biotechnology, 104-108 (1992)) using the AmpliTaq DNA Polymerase FS kit and 373A DNA Sequencer Stretch (PE Applied Biosystems, Mississauga, ON, Canada). The host bacteria used for cloning was TG1: supE hsdδ thi (lac-proAB) F [fraD36 proAB+ lacP/acZM15]. All the cloning steps were performed as described (Sambrook et al., supra).

### BRIEF DESCRIPTION OF FIGURES AND TABLES

FIG. 1. The T-cell receptor complex with TCR-α and TCR-β chains (top), ζ-chain accessory molecules (bottom) and CD3 (represented by CD3γ, CD3δ and two CD3ε).

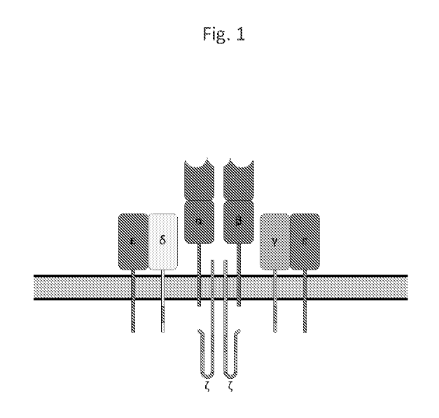

FIG. 2. Schematic representation of conventional and camelid antibodies and SDS/PAGE analysis in reducing and non-reducing condition of camelid antibodies.

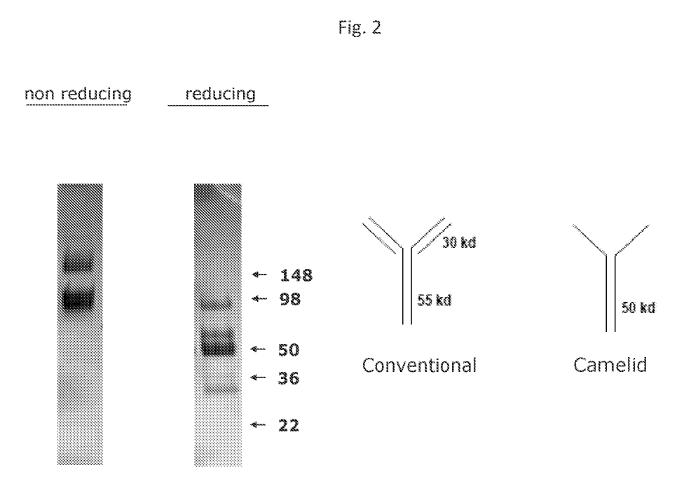

FIG. 3. Time course detection of CD3ε specific antibody titers in serum of the immunized camel. Human recombinant CD80 was used as a negative control.

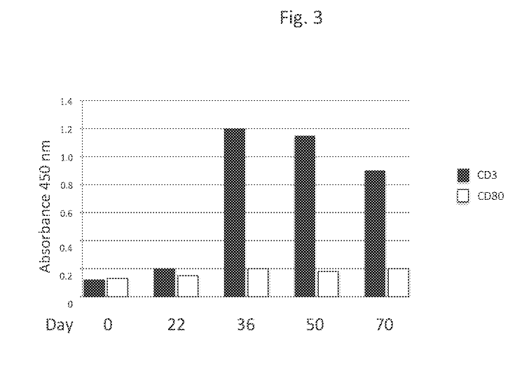

FIG. 4. Amplification and cloning of the VHH encoding cDNAs from the immunized camel.

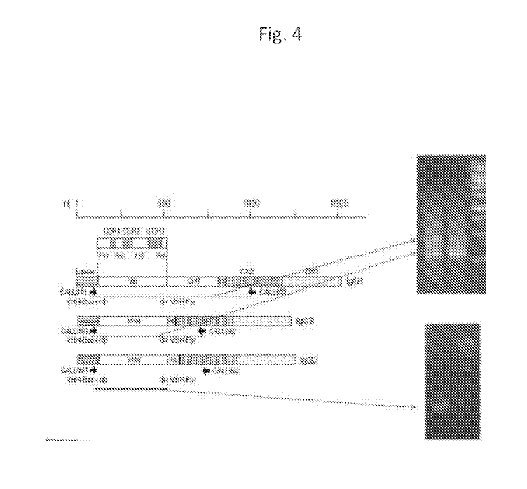

FIG. 5. Overview of selection procedure used to identify CD3ε-binding VHHs.

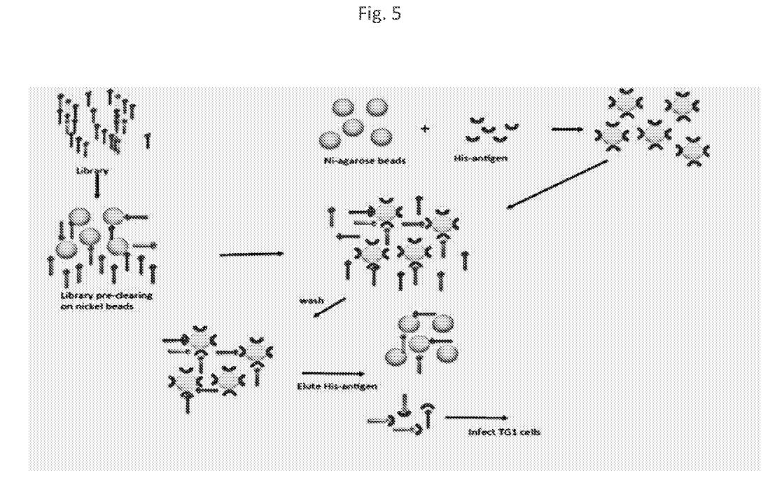

FIG. 6. One-step enrichment for CD3ε binding VHHs.

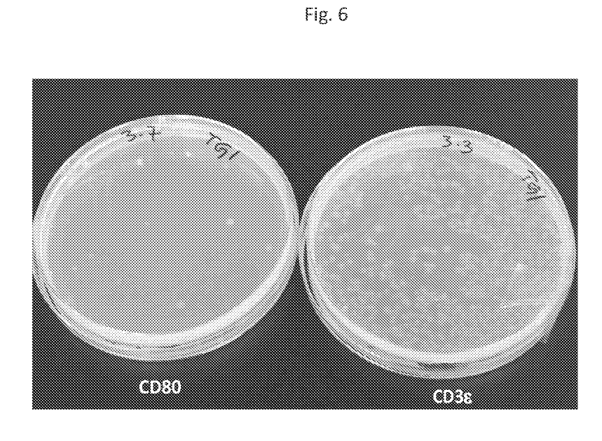

FIG. 7. Amino acid alignment of the CDR regions of 20 clones identified by the epitope specific elution selection procedure (SEQ ID NOS 43-102, respectively, in order of appearance)”).

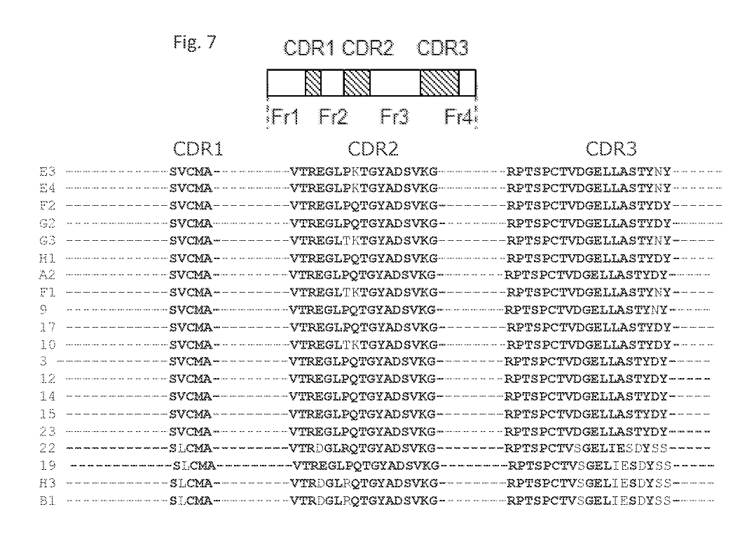

FIG. 8. ELISA assay on solid-phase immobilized CD3ε of 4 unique CD3ε specific clones identified via the epitope specific elution selection procedure. 2 VHH clones randomly picked from a preimmune library were used as a control.

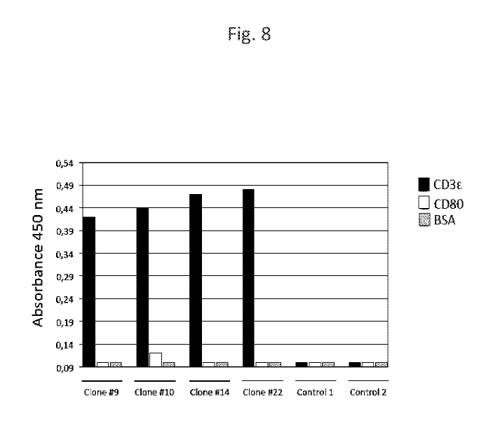

FIG. 9. Pull down assay on CD3ε of 4 unique CD3ε specific clones identified via the epitope specific elution selection procedure. 2 VHH clones randomly picked from a preimmune library were used as a control.

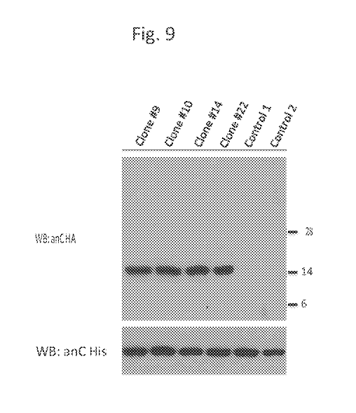

FIG. 10. Western Blot. Cell lysates were prepared from Jurkat cells in lysis buffer (150 mM NaCl, 20 mM Hepes, pH 7.4, 1% Triton X-100, 10% glycerol and a mixture of protease inhibitors). Proteins were separated by SDS-PAGE, transferred onto nitrocellulose membrane and incubated with 4 CD3ε-specific VHH or 2 VHH randomly picked from a preimmune library primary antibodies followed by anti-HA and horseradish peroxidase-conjugated secondary antibodies (Amersham Biosciences). The blot was developed with SuperSignal West™ substrate (Pierce) and exposed to film as shown in the Figure.

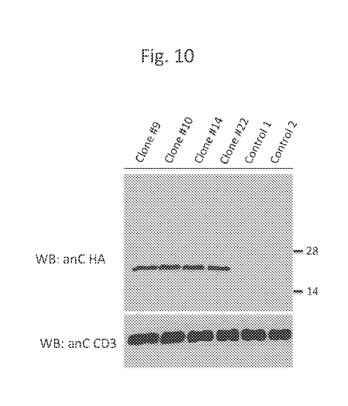

Table I Amino acid sequence listing of the peptides of the present invention.

FIG. 11. Table II. The extracellular portion of the CD3ε antigen expressed on human T cells used to raise an antibody response in camels (SEQ. ID. NO: 103).

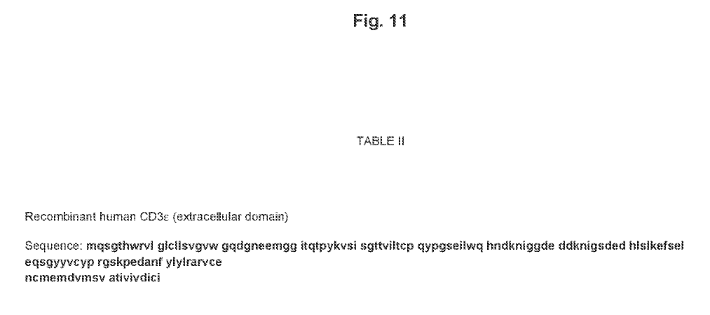

### QUOTED PATENTS

| | | | | |
|---|---|---|---|---|
| U.S. Pat. No. | 6 Jun. 1995 | 2 Jun. 1998 | Vrije | Immunoglobulins |
| 5,759,808 | | | Universiteit | devoid of light chains |
| | | | Brussels | |
| U.S. Pat. No. | 6 Jun. 1995 | 1 Sep. 1998 | Vrije | Immunoglobulins |
| 5,800,988 | | | Universiteit | devoid of light chains |
| | | | Brussels | |
| U.S. Pat. No. | 6 Jun. 1995 | 24 Nov 1998 | Vrije | Immunoglobulins |
| 5,840,526 | | | Universiteit | devoid of light chains |
| | | | Brussels | |
| U.S. Pat. No. | 6 Jun. 1995 | 23 Feb. 1999 | Vrije | Immunoglobulins |
| 5,874,541 | | | Universiteit | devoid of light chains |
| WO1994004678A1 | 18 Aug. 1993 | 3 Mar. 1994 | Casterman | Immunoglobulins |
| | | | Cecile | devoid of light chains |
| WO1997049805 | 27 Jun. 1997 | 31 Dec. 1997 | Serge | Recognition molecules |
| | | | Muyldermans, | interacting specifically |
| | | | Lode Wyns | with the active site or |
| | | | | cleft of a target |
| | | | | molecule |
| WO 1996034103 | 25 Apr. 1996 | 31 Oct. 1997 | Raymond | Variable fragments of |
| | | | Hamers, Serge | immunoglobulins - use |
| | | | Muyldermans | for therapeutic or |
| | | | | veterinary purposes |
| EP0707473A1 | 2 Jun. 1994 | 24 Apr. 1996 | Biotechnology | Oral pharmaceutical |
| | | | and Biological | compositions |
| | | | Sciences | comprising a protein or |
| | | | Research | peptide, an antibody |
| | | | Council | and polymeric beads |
| EP0684814A1 | 22 Feb. 1994 | 6 Dec. 1995 | Alza | Compositions for oral |
| | | | Corporation | delivery of active |
| | | | | agents |
| U.S. Pat. No. | 30 Jan. 1995 | 13 May. 1997 | University | Oral administration of |
| 5,629,001 | | | Of Cincinnati | therapeutic proteins for |
| | | | | treatment of infectious |
| | | | | disease |
| U.S. Pat. No. | 28 Aug. 1984 | 26 Aug. 1986 | Societe | Method for producing a |
| 4,608,392 | | | Anonyme | non-greasy protective |
| | | | Dite: L'oreal | and emollient film on |
| | | | | the skin |
| U.S. Pat. No. | 23 giu 1987 | 11 Apr. 1989 | Neutrogena | Skin protective |
| 4,820,508 | | | Corporation | composition |
| U.S. Pat. No. | 21 Apr. 1983 | 17 Dec. 1985 | Creative | Cosmetic applicator |
| 4,559,157 | | | Products | useful for skin |
| | | | Resource | moisturizing |
| | | | Associates, | |
| | | | Ltd. | |
| U.S. Pat. No. | 4 Apr. 1988 | 12 Feb. 1991 | Warner- | Anti-inflammatory skin |
| 4,992,478 | | | Lambert | moisturizing |
| | | | Company | composition and |
| | | | | method of preparing |
| | | | | same |

TABLE I
SEQ. ID 1
MAESGGGSVQ TGGSLRLSCA YTASSVCMAW
FRQAPGKERE GVAVTREGLT KTGYADSVKG
RFAISQDYAK KTLYLQMSSL KPEDTARYYC
AARPTSPCTV DGELLASTYN YWGQGTQVTV
SEQ. ID 2
MAESGGGSVQ TGGSLRLSCA YTASSVCMAW
FRQAPGKERE GVAVTREGLT KTGYADSVKG
RFAISQDYAK KTLYLQMSSL KPEDTARYYC
AARPTSPCTV DGELLASTYD YWGQGTQVTV
SEQ. ID 3
MAESGGGSVQ TGGSLRLSCA YTASSVCMAW
FRQAPGKERE GVAVTREGLT QTGYADSVKG
RFAISQDYAK KTLYLQMSSL KPEDTARYYC
AARPTSPCTV DGELLASTYN YWGQGTQVTV
SEQ. ID 4
MAESGGGSVQ TGGSLRLSCA YTASSVCMAW
FRQAPGKERE GVAVTREGLT QTGYADSVKG
RFAISQDYAK KTLYLQMSSL KPEDTARYYC
AARPTSPCTV DGELLASTYD YWGQGTQVTV
SEQ. ID 5
MAESGGGSVQ TGGSLRLSCA YTASSVCMAW
FRQAPGKERE GVAVTREGLP KTGYADSVKG
RFAISQDYAK KTLYLQMSSL KPEDTARYYC
AARPTSPCTV DGELLASTYN YWGQGTQVTV
SEQ. ID 6
MAESGGGSVQ TGGSLRLSCA YTASSVCMAW
FRQAPGKERE GVAVTREGLP KTGYADSVKG
RFAISQDYAK KTLYLQMSSL KPEDTARYYC
AARPTSPCTV DGELLASTYD YWGQGTQVTV
SEQ. ID 7
MAESGGGSVQ TGGSLRLSCA YTASSVCMAW
FRQAPGKERE GVAVTREGLP QTGYADSVKG
RFAISQDYAK KTLYLQMSSL KPEDTARYYC
AARPTSPCTV DGELLASTYN YWGQGTQVTV
SEQ. ID 8
MAESGGGSVQ TGGSLRLSCA YTASSVCMAW
FRQAPGKERE GVAVTREGLP QTGYADSVKG
RFAISQDYAK KTLYLQMSSL KPEDTARYYC
AARPTSPCTV DGELLASTYD YWGQGTQVTV
SEQ. ID 9
MAESGGGSVQ TGGSLRLSCA YTASSVCMAW
FRQAPGKERE GVAVTRDGLT KTGYADSVKG
RFAISQDYAK KTLYLQMSSL KPEDTARYYC
AARPTSPCTV DGELLASTYN YWGQGTQVTV
SEQ. ID 10
MAESGGGSVQ TGGSLRLSCA YTASSVCMAW
FRQAPGKERE GVAVTRDGLT KTGYADSVKG
RFAISQDYAK KTLYLQMSSL KPEDTARYYC
AARPTSPCTV DGELLASTYD YWGQGTQVTV
SEQ. ID 11
MAESGGGSVQ TGGSLRLSCA YTASSVCMAW
FRQAPGKERE GVAVTRDGLT QTGYADSVKG
RFAISQDYAK KTLYLQMSSL KPEDTARYYC
AARPTSPCTV DGELLASTYN YWGQGTQVTV
SEQ. ID 12
MAESGGGSVQ TGGSLRLSCA YTASSVCMAW
FRQAPGKERE GVAVTRDGLT QTGYADSVKG
RFAISQDYAK KTLYLQMSSL KPEDTARYYC
AARPTSPCTV DGELLASTYD YWGQGTQVTV
SEQ. ID 13
MAESGGGSVQ TGGSLRLSCA YTASSVCMAW
FRQAPGKERE GVAVTRDGLP KTGYADSVKG
RFAISQDYAK KTLYLQMSSL KPEDTARYYC
AARPTSPCTV DGELLASTYN YWGQGTQVTV
SEQ. ID 14
MAESGGGSVQ TGGSLRLSCA YTASSVCMAW
FRQAPGKERE GVAVTRDGLP KTGYADSVKG
RFAISQDYAK KTLYLQMSSL KPEDTARYYC
AARPTSPCTV DGELLASTYD YWGQGTQVTV
SEQ. ID 15
MAESGGGSVQ TGGSLRLSCA YTASSVCMAW
FRQAPGKERE GVAVTRDGLP QTGYADSVKG
RFAISQDYAK KTLYLQMSSL KPEDTARYYC
AARPTSPCTV DGELLASTYN YWGQGTQVTV
SEQ. ID 16
MAESGGGSVQ TGGSLRLSCA YTASSVCMAW
FRQAPGKERE GVAVTRDGLP QTGYADSVKG
RFAISQDYAK KTLYLQMSSL KPEDTARYYC
AARPTSPCTV DGELLASTYD YWGQGTQVTV
SEQ. ID 17
MAESGGGSVQ TGGSLRLSCA YTASSLCMAW
FRQAPGKERE GVAVTREGLT KTGYADSVKG
RFAISQDYAK KTLYLQMSSL KPEDTARYYC
AARPTSPCTV DGELLASTYN YWGQGTQVTV
SEQ. ID 18
MAESGGGSVQ TGGSLRLSCA YTASSLCMAW
FRQAPGKERE GVAVTREGLT KTGYADSVKG
RFAISQDYAK KTLYLQMSSL KPEDTARYYC
AARPTSPCTV DGELLASTYD YWGQGTQVTV
SEQ. ID 19
MAESGGGSVQ TGGSLRLSCA YTASSLCMAW
FRQAPGKERE GVAVTREGLT QTGYADSVKG
RFAISQDYAK KTLYLQMSSL KPEDTARYYC
AARPTSPCTV DGELLASTYN YWGQGTQVTV
SEQ. ID 20
MAESGGGSVQ TGGSLRLSCA YTASSLCMAW
FRQAPGKERE GVAVTREGLT QTGYADSVKG
RFAISQDYAK KTLYLQMSSL KPEDTARYYC
AARPTSPCTV DGELLASTYD YWGQGTQVTV
SEQ. ID 21
MAESGGGSVQ TGGSLRLSCA YTASSLCMAW
FRQAPGKERE GVAVTREGLP KTGYADSVKG
RFAISQDYAK KTLYLQMSSL KPEDTARYYC
AARPTSPCTV DGELLASTYN YWGQGTQVTV
SEQ. ID 22
MAESGGGSVQ TGGSLRLSCA YTASSLCMAW
FRQAPGKERE GVAVTREGLP KTGYADSVKG
RFAISQDYAK KTLYLQMSSL KPEDTARYYC
AARPTSPCTV DGELLASTYD YWGQGTQVTV
SEQ. ID 23
MAESGGGSVQ TGGSLRLSCA YTASSLCMAW
FRQAPGKERE GVAVTREGLP QTGYADSVKG
RFAISQDYAK KTLYLQMSSL KPEDTARYYC
AARPTSPCTV DGELLASTYN YWGQGTQVTV
SEQ. ID 24
MAESGGGSVQ TGGSLRLSCA YTASSLCMAW
FRQAPGKERE GVAVTREGLP QTGYADSVKG
RFAISQDYAK KTLYLQMSSL KPEDTARYYC
AARPTSPCTV DGELLASTYD YWGQGTQVTV
SEQ. ID 25
MAESGGGSVQ TGGSLRLSCA YTASSLCMAW
FRQAPGKERE GVAVTRDGLT KTGYADSVKG
RFAISQDYAK KTLYLQMSSL KPEDTARYYC
AARPTSPCTV DGELLASTYN YWGQGTQVTV
SEQ. ID 26
MAESGGGSVQ TGGSLRLSCA YTASSLCMAW
FRQAPGKERE GVAVTRDGLT KTGYADSVKG
RFAISQDYAK KTLYLQMSSL KPEDTARYYC
AARPTSPCTV DGELLASTYD YWGQGTQVTV
SEQ. ID 27
MAESGGGSVQ TGGSLRLSCA YTASSLCMAW
FRQAPGKERE GVAVTRDGLT QTGYADSVKG
RFAISQDYAK KTLYLQMSSL KPEDTARYYC
AARPTSPCTV DGELLASTYN YWGQGTQVTV
SEQ. ID 28
MAESGGGSVQ TGGSLRLSCA YTASSLCMAW
FRQAPGKERE GVAVTRDGLT QTGYADSVKG
RFAISQDYAK KTLYLQMSSL KPEDTARYYC
AARPTSPCTV DGELLASTYD YWGQGTQVTV
SEQ. ID 29
MAESGGGSVQ TGGSLRLSCA YTASSLCMAW
FRQAPGKERE GVAVTRDGLP KTGYADSVKG
RFAISQDYAK KTLYLQMSSL KPEDTARYYC
AARPTSPCTV DGELLASTYN YWGQGTQVTV
SEQ. ID 30
MAESGGGSVQ TGGSLRLSCA YTASSLCMAW
FRQAPGKERE GVAVTRDGLP KTGYADSVKG
RFAISQDYAK KTLYLQMSSL KPEDTARYYC
AARPTSPCTV DGELLASTYD YWGQGTQVTV
SEQ. ID 31
MAESGGGSVQ TGGSLRLSCA YTASSLCMAW
FRQAPGKERE GVAVTRDGLP QTGYADSVKG
RFAISQDYAK KTLYLQMSSL KPEDTARYYC
AARPTSPCTV DGELLASTYN YWGQGTQVTV
SEQ. ID 32
MAESGGGSVQ TGGSLRLSCA YTASSLCMAW
FRQAPGKERE GVAVTRDGLP QTGYADSVKG
RFAISQDYAK KTLYLQMSSL KPEDTARYYC
AARPTSPCTV DGELLASTYD YWGQGTQVTV
SEQ. ID 33
GQHHHHHH
SEQ. ID 34
MAESGGGSVQ TGGSLRLSCA YTASSVCMAW
FRQAPGKERE GVAVTREGLT KTGYADSVKG
RFAISQDYAK KTLYLQMSSL KPEDTARYYC
AARPTSPCTV DGELLASTYN YWGQGTQVTV
GQHHHHHH
SEQ. ID 35
MAESGGGSVQ TGGSLRLSCA YTASSVCMAW
FRQAPGKERE GVAVTREGLT KTGYADSVKG
RFAISQDYAK KTLYLQMSSL KPEDTARYYC
AARPTSPCTV DGELLASTYD YWGQGTQVTV
GQHHHHHH
SEQ. ID 36
MAESGGGSVQ TGGSLRLSCA YTASSVCMAW
FRQAPGKERE GVAVTREGLT QTGYADSVKG
RFAISQDYAK KTLYLQMSSL KPEDTARYYC
AARPTSPCTV DGELLASTYN YWGQGTQVTV
GQHHHHHH
CALL001:
SEQ. ID 37
5′-GTCCTGGCTGCTCTTCTACAAGG-3′
CALL002:
SEQ. ID 38
5′-GGTACGTGCTGTTGAACTGTTCC-3′
VHH-Back:
SEQ. ID 39
5′-GATGTGCAGCTGCAGGAGTCTGGRGGAGG-3′
VHH-For:
SEQ. ID 40
5′-CTAGTGCGGCCGCTGGAGACGGTGACCTGGGT-3′

The invention claimed is:

1. An isolated variable domain of a camelid heavy-chain antibody (VHH domain) directed against CD3epslion (CD3ε) component of the T cell receptor, wherein said VHH domain comprises an amino acid sequence selected from the group consisting amino acid sequence SEQ ID NO: 1 to SEQ ID NO: 32.

2. A VHH domain according to claim 1, comprising a homo/hetero dimers of SEQ ID NO: 1 to SEQ ID NO: 32

3. A VHH domain according to claim 1, wherein it is obtainable by the method comprising the steps of: (a) immunizing a camelid with recombinant CD3ε, (b) isolating peripheral lymphocytes of the immunized camelid, obtaining the total RNA and synthesizing the corresponding cDNAs, (c) constructing a library of cDNA fragments encoding VHH domains, (d) transcribing the VHH domain-encoding cDNAs obtained in step (c) to mRNA using PCR, converting the mRNA to phage display format, and selecting the VHH domain by phage display, and (e) expressing the VHH domain in a vector.

4. An isolated polypeptide, comprising a VHH domain according to claim 1.

5. The polypeptide according to claim 4, wherein it contains at its C-terminus the amino acid sequence LEHHHHHH (SEQ ID NO: 42).

6. An isolated antibody or fragment thereof, comprising a VHH domain according to claim 1.

7. A therapeutic or diagnostic agent, comprising a VHH domain according to claim 1, linked, directly or indirectly, covalently or non-covalently to a substance of interest.

8. A therapeutic or diagnostic agent according to claim 7, wherein said substance of interest is a therapeutic or diagnostic compound selected from the group consisting of a peptide, an enzyme, a virus, a fluorophore, a heavy metal, a chemical entity and a radioisotope.

9. A therapeutic or diagnostic agent according to claim 8, wherein said substance of interest is a liposome or a polymeric entity comprising a therapeutic or a diagnostic compound selected from the group consisting of a peptide, an enzyme, a virus, a fluorophore, a heavy metal, a chemical entity and a radioisotope.

10. A therapeutic or diagnostic agent according to claim 8, wherein said therapeutic compound is selected from the group consisting of an anticancer compound, an immunosuppressant compound and an anti-inflammatory compound.

11. A therapeutic or diagnostic agent, comprising a polypeptide according to claim 4, linked, directly or indirectly, covalently or non-covalently to a substance of interest.

12. A therapeutic or diagnostic agent, comprising an antibody according to claim 6, linked, directly or indirectly, covalently or non-covalently to a substance of interest.

13. A kit comprising a VHH domain according to claim 1.

14. A kit comprising a polypeptide according to claim 4.

15. A kit comprising an antibody according to claim 6.

16. A kit comprising a diagnostic agent according to claim 7.

17. A pharmaceutical composition, comprising a therapeutic agent according to claim 7 and a pharmaceutically acceptable carrier.

18. A method of indicating the presence of a disorder mediated by infiltrating T lymphocytes comprising the steps of: a) contacting in vitro or ex vivo an appropriate biological sample with a VHH domain according to claim 1, b) determining the amount of CD3ε in said biological sample, and c) comparing the amount determined in step (b) with a standard, a difference in amount indicating the presence of said disorder.

19. The method of claim 18, wherein said polypeptide contains at its C-terminus the amino acid sequence GQHHHHHH (SEQ ID NO: 33).


SEQUENCE LISTING (103 sequences)

SEQ ID NO: 1 - protein, 120 aa, Artificial Sequence
  Description of Artificial Sequence Synthetic polypeptide
MAESGGGSVQTGGSLRLSCAYTASSVCMAWFRQAPGKEREGVAVTREGLTKTGYADSVKGRFAISQDYAKKTLYLQMSSLKPEDTARYYCAARPTSPCTVDGELLASTYNYWGQGTQVTV

SEQ ID NO: 2 - protein, 120 aa, Artificial Sequence
  Description of Artificial Sequence Synthetic polypeptide
MAESGGGSVQTGGSLRLSCAYTASSVCMAWFRQAPGKEREGVAVTREGLTKTGYADSVKGRFAISQDYAKKTLYLQMSSLKPEDTARYYCAARPTSPCTVDGELLASTYDYWGQGTQVTV

SEQ ID NO: 3 - protein, 120 aa, Artificial Sequence
  Description of Artificial Sequence Synthetic polypeptide
MAESGGGSVQTGGSLRLSCAYTASSVCMAWFRQAPGKEREGVAVTREGLTQTGYADSVKGRFAISQDYAKKTLYLQMSSLKPEDTARYYCAARPTSPCTVDGELLASTYNYWGQGTQVTV

SEQ ID NO: 4 - protein, 120 aa, Artificial Sequence
  Description of Artificial Sequence Synthetic polypeptide
MAESGGGSVQTGGSLRLSCAYTASSVCMAWFRQAPGKEREGVAVTREGLTQTGYADSVKGRFAISQDYAKKTLYLQMSSLKPEDTARYYCAARPTSPCTVDGELLASTYDYWGQGTQVTV

SEQ ID NO: 5 - protein, 120 aa, Artificial Sequence
  Description of Artificial Sequence Synthetic polypeptide
MAESGGGSVQTGGSLRLSCAYTASSVCMAWFRQAPGKEREGVAVTREGLPKTGYADSVKGRFAISQDYAKKTLYLQMSSLKPEDTARYYCAARPTSPCTVDGELLASTYNYWGQGTQVTV

SEQ ID NO: 6 - protein, 120 aa, Artificial Sequence
  Description of Artificial Sequence Synthetic polypeptide
MAESGGGSVQTGGSLRLSCAYTASSVCMAWFRQAPGKEREGVAVTREGLPKTGYADSVKGRFAISQDYAKKTLYLQMSSLKPEDTARYYCAARPTSPCTVDGELLASTYDYWGQGTQVTV

SEQ ID NO: 7 - protein, 120 aa, Artificial Sequence
  Description of Artificial Sequence Synthetic polypeptide
MAESGGGSVQTGGSLRLSCAYTASSVCMAWFRQAPGKEREGVAVTREGLPQTGYADSVKGRFAISQDYAKKTLYLQMSSLKPEDTARYYCAARPTSPCTVDGELLASTYNYWGQGTQVTV

SEQ ID NO: 8 - protein, 120 aa, Artificial Sequence
  Description of Artificial Sequence Synthetic polypeptide
MAESGGGSVQTGGSLRLSCAYTASSVCMAWFRQAPGKEREGVAVTREGLPQTGYADSVKGRFAISQDYAKKTLYLQMSSLKPEDTARYYCAARPTSPCTVDGELLASTYDYWGQGTQVTV

SEQ ID NO: 9 - protein, 120 aa, Artificial Sequence
  Description of Artificial Sequence Synthetic polypeptide
MAESGGGSVQTGGSLRLSCAYTASSVCMAWFRQAPGKEREGVAVTRDGLTKTGYADSVKGRFAISQDYAKKTLYLQMSSLKPEDTARYYCAARPTSPCTVDGELLASTYNYWGQGTQVTV

SEQ ID NO: 10 - protein, 120 aa, Artificial Sequence
  Description of Artificial Sequence Synthetic polypeptide
MAESGGGSVQTGGSLRLSCAYTASSVCMAWFRQAPGKEREGVAVTRDGLTKTGYADSVKGRFAISQDYAKKTLYLQMSSLKPEDTARYYCAARPTSPCTVDGELLASTYDYWGQGTQVTV

SEQ ID NO: 11 - protein, 120 aa, Artificial Sequence
  Description of Artificial Sequence Synthetic polypeptide
MAESGGGSVQTGGSLRLSCAYTASSVCMAWFRQAPGKEREGVAVTRDGLTQTGYADSVKGRFAISQDYAKKTLYLQMSSLKPEDTARYYCAARPTSPCTVDGELLASTYNYWGQGTQVTV

SEQ ID NO: 12 - protein, 120 aa, Artificial Sequence
  Description of Artificial Sequence Synthetic polypeptide
MAESGGGSVQTGGSLRLSCAYTASSVCMAWFRQAPGKEREGVAVTRDGLTQTGYADSVKGRFAISQDYAKKTLYLQMSSLKPEDTARYYCAARPTSPCTVDGELLASTYDYWGQGTQVTV

SEQ ID NO: 13 - protein, 120 aa, Artificial Sequence
  Description of Artificial Sequence Synthetic polypeptide
MAESGGGSVQTGGSLRLSCAYTASSVCMAWFRQAPGKEREGVAVTRDGLPKTGYADSVKGRFAISQDYAKKTLYLQMSSLKPEDTARYYCAARPTSPCTVDGELLASTYNYWGQGTQVTV

SEQ ID NO: 14 - protein, 120 aa, Artificial Sequence
  Description of Artificial Sequence Synthetic polypeptide
MAESGGGSVQTGGSLRLSCAYTASSVCMAWFRQAPGKEREGVAVTRDGLPKTGYADSVKGRFAISQDYAKKTLYLQMSSLKPEDTARYYCAARPTSPCTVDGELLASTYDYWGQGTQVTV

SEQ ID NO: 15 - protein, 120 aa, Artificial Sequence
  Description of Artificial Sequence Synthetic polypeptide
MAESGGGSVQTGGSLRLSCAYTASSVCMAWFRQAPGKEREGVAVTRDGLPQTGYADSVKGRFAISQDYAKKTLYLQMSSLKPEDTARYYCAARPTSPCTVDGELLASTYNYWGQGTQVTV

SEQ ID NO: 16 - protein, 120 aa, Artificial Sequence
  Description of Artificial Sequence Synthetic polypeptide
MAESGGGSVQTGGSLRLSCAYTASSVCMAWFRQAPGKEREGVAVTRDGLPQTGYADSVKGRFAISQDYAKKTLYLQMSSLKPEDTARYYCAARPTSPCTVDGELLASTYDYWGQGTQVTV

SEQ ID NO: 17 - protein, 120 aa, Artificial Sequence
  Description of Artificial Sequence Synthetic polypeptide
MAESGGGSVQTGGSLRLSCAYTASSLCMAWFRQAPGKEREGVAVTREGLTKTGYADSVKGRFAISQDYAKKTLYLQMSSLKPEDTARYYCAARPTSPCTVDGELLASTYNYWGQGTQVTV

SEQ ID NO: 18 - protein, 120 aa, Artificial Sequence
  Description of Artificial Sequence Synthetic polypeptide
MAESGGGSVQTGGSLRLSCAYTASSLCMAWFRQAPGKEREGVAVTREGLTKTGYADSVKGRFAISQDYAKKTLYLQMSSLKPEDTARYYCAARPTSPCTVDGELLASTYDYWGQGTQVTV

SEQ ID NO: 19 - protein, 120 aa, Artificial Sequence
  Description of Artificial Sequence Synthetic polypeptide
MAESGGGSVQTGGSLRLSCAYTASSLCMAWFRQAPGKEREGVAVTREGLTQTGYADSVKGRFAISQDYAKKTLYLQMSSLKPEDTARYYCAARPTSPCTVDGELLASTYNYWGQGTQVTV

SEQ ID NO: 20 - protein, 120 aa, Artificial Sequence
  Description of Artificial Sequence Synthetic polypeptide
MAESGGGSVQTGGSLRLSCAYTASSLCMAWFRQAPGKEREGVAVTREGLTQTGYADSVKGRFAISQDYAKKTLYLQMSSLKPEDTARYYCAARPTSPCTVDGELLASTYDYWGQGTQVTV

SEQ ID NO: 21 - protein, 120 aa, Artificial Sequence
  Description of Artificial Sequence Synthetic polypeptide
MAESGGGSVQTGGSLRLSCAYTASSLCMAWFRQAPGKEREGVAVTREGLPKTGYADSVKGRFAISQDYAKKTLYLQMSSLKPEDTARYYCAARPTSPCTVDGELLASTYNYWGQGTQVTV

SEQ ID NO: 22 - protein, 120 aa, Artificial Sequence
  Description of Artificial Sequence Synthetic polypeptide
MAESGGGSVQTGGSLRLSCAYTASSLCMAWFRQAPGKEREGVAVTREGLPKTGYADSVKGRFAISQDYAKKTLYLQMSSLKPEDTARYYCAARPTSPCTVDGELLASTYDYWGQGTQVTV

SEQ ID NO: 23 - protein, 120 aa, Artificial Sequence
  Description of Artificial Sequence Synthetic polypeptide
MAESGGGSVQTGGSLRLSCAYTASSLCMAWFRQAPGKEREGVAVTREGLPQTGYADSVKGRFAISQDYAKKTLYLQMSSLKPEDTARYYCAARPTSPCTVDGELLASTYNYWGQGTQVTV

SEQ ID NO: 24 - protein, 120 aa, Artificial Sequence
  Description of Artificial Sequence Synthetic polypeptide
MAESGGGSVQTGGSLRLSCAYTASSLCMAWFRQAPGKEREGVAVTREGLPQTGYADSVKGRFAISQDYAKKTLYLQMSSLKPEDTARYYCAARPTSPCTVDGELLASTYDYWGQGTQVTV

SEQ ID NO: 25 - protein, 120 aa, Artificial Sequence
  Description of Artificial Sequence Synthetic polypeptide
MAESGGGSVQTGGSLRLSCAYTASSLCMAWFRQAPGKEREGVAVTRDGLTKTGYADSVKGRFAISQDYAKKTLYLQMSSLKPEDTARYYCAARPTSPCTVDGELLASTYNYWGQGTQVTV

SEQ ID NO: 26 - protein, 120 aa, Artificial Sequence
  Description of Artificial Sequence Synthetic polypeptide
MAESGGGSVQTGGSLRLSCAYTASSLCMAWFRQAPGKEREGVAVTRDGLTKTGYADSVKGRFAISQDYAKKTLYLQMSSLKPEDTARYYCAARPTSPCTVDGELLASTYDYWGQGTQVTV

SEQ ID NO: 27 - protein, 120 aa, Artificial Sequence
  Description of Artificial Sequence Synthetic polypeptide
MAESGGGSVQTGGSLRLSCAYTASSLCMAWFRQAPGKEREGVAVTRDGLTQTGYADSVKGRFAISQDYAKKTLYLQMSSLKPEDTARYYCAARPTSPCTVDGELLASTYNYWGQGTQVTV

SEQ ID NO: 28 - protein, 120 aa, Artificial Sequence
  Description of Artificial Sequence Synthetic polypeptide
MAESGGGSVQTGGSLRLSCAYTASSLCMAWFRQAPGKEREGVAVTRDGLTQTGYADSVKGRFAISQDYAKKTLYLQMSSLKPEDTARYYCAARPTSPCTVDGELLASTYDYWGQGTQVTV

SEQ ID NO: 29 - protein, 120 aa, Artificial Sequence
  Description of Artificial Sequence Synthetic polypeptide
MAESGGGSVQTGGSLRLSCAYTASSLCMAWFRQAPGKEREGVAVTRDGLPKTGYADSVKGRFAISQDYAKKTLYLQMSSLKPEDTARYYCAARPTSPCTVDGELLASTYNYWGQGTQVTV

SEQ ID NO: 30 - protein, 120 aa, Artificial Sequence
  Description of Artificial Sequence Synthetic polypeptide
MAESGGGSVQTGGSLRLSCAYTASSLCMAWFRQAPGKEREGVAVTRDGLPKTGYADSVKGRFAISQDYAKKTLYLQMSSLKPEDTARYYCAARPTSPCTVDGELLASTYDYWGQGTQVTV

SEQ ID NO: 31 - protein, 120 aa, Artificial Sequence
  Description of Artificial Sequence Synthetic polypeptide
MAESGGGSVQTGGSLRLSCAYTASSLCMAWFRQAPGKEREGVAVTRDGLPQTGYADSVKGRFAISQDYAKKTLYLQMSSLKPEDTARYYCAARPTSPCTVDGELLASTYNYWGQGTQVTV

SEQ ID NO: 32 - protein, 120 aa, Artificial Sequence
  Description of Artificial Sequence Synthetic polypeptide
MAESGGGSVQTGGSLRLSCAYTASSLCMAWFRQAPGKEREGVAVTRDGLPQTGYADSVKGRFAISQDYAKKTLYLQMSSLKPEDTARYYCAARPTSPCTVDGELLASTYDYWGQGTQVTV

SEQ ID NO: 33 - protein, 8 aa, Artificial Sequence
  Description of Artificial Sequence Synthetic peptide
GQHHHHHH

SEQ ID NO: 34 - protein, 128 aa, Artificial Sequence
  Description of Artificial Sequence Synthetic polypeptide
MAESGGGSVQTGGSLRLSCAYTASSVCMAWFRQAPGKEREGVAVTREGLTKTGYADSVKGRFAISQDYAKKTLYLQMSSLKPEDTARYYCAARPTSPCTVDGELLASTYNYWGQGTQVTVGQHHHHHH

SEQ ID NO: 35 - protein, 128 aa, Artificial Sequence
  Description of Artificial Sequence Synthetic polypeptide
MAESGGGSVQTGGSLRLSCAYTASSVCMAWFRQAPGKEREGVAVTREGLTKTGYADSVKGRFAISQDYAKKTLYLQMSSLKPEDTARYYCAARPTSPCTVDGELLASTYDYWGQGTQVTVGQHHHHHH

SEQ ID NO: 36 - protein, 128 aa, Artificial Sequence
  Description of Artificial Sequence Synthetic polypeptide
MAESGGGSVQTGGSLRLSCAYTASSVCMAWFRQAPGKEREGVAVTREGLTQTGYADSVKGRFAISQDYAKKTLYLQMSSLKPEDTARYYCAARPTSPCTVDGELLASTYNYWGQGTQVTVGQHHHHHH

SEQ ID NO: 37 - DNA, 23 bp, Artificial Sequence
  Description of Artificial Sequence Synthetic primer
gtcctggctgctcttctacaagg

SEQ ID NO: 38 - DNA, 23 bp, Artificial Sequence
  Description of Artificial Sequence Synthetic primer
ggtacgtgctgttgaactgttcc

SEQ ID NO: 39 - DNA, 29 bp, Artificial Sequence
  Description of Artificial Sequence Synthetic primer
gatgtgcagctgcaggagtctggrggagg

SEQ ID NO: 40 - DNA, 32 bp, Artificial Sequence
  Description of Artificial Sequence Synthetic primer
ctagtgcggccgctggagacggtgacctgggt

SEQ ID NO: 41 - protein, 6 aa, Artificial Sequence
  Description of Artificial Sequence Synthetic 6xHis tag
HHHHHH

SEQ ID NO: 42 - protein, 8 aa, Artificial Sequence
  Description of Artificial Sequence Synthetic peptide
LEHHHHHH

SEQ ID NO: 43 - protein, 5 aa, Artificial Sequence
  Description of Artificial Sequence Synthetic peptide
SVCMA

SEQ ID NO: 44 - protein, 17 aa, Artificial Sequence
  Description of Artificial Sequence Synthetic peptide
VTREGLPKTGYADSVKG

SEQ ID NO: 45 - protein, 19 aa, Artificial Sequence
  Description of Artificial Sequence Synthetic peptide
RPTSPCTVDGELLASTYNY

SEQ ID NO: 46 - protein, 5 aa, Artificial Sequence
  Description of Artificial Sequence Synthetic peptide
SVCMA

SEQ ID NO: 47 - protein, 17 aa, Artificial Sequence
  Description of Artificial Sequence Synthetic peptide
VTREGLPKTGYADSVKG

SEQ ID NO: 48 - protein, 19 aa, Artificial Sequence
  Description of Artificial Sequence Synthetic peptide
RPTSPCTVDGELLASTYNY

SEQ ID NO: 49 - protein, 5 aa, Artificial Sequence
  Description of Artificial Sequence Synthetic peptide
SVCMA

SEQ ID NO: 50 - protein, 17 aa, Artificial Sequence
  Description of Artificial Sequence Synthetic peptide
VTREGLPQTGYADSVKG

SEQ ID NO: 51 - protein, 19 aa, Artificial Sequence
  Description of Artificial Sequence Synthetic peptide
RPTSPCTVDGELLASTYDY

SEQ ID NO: 52 - protein, 5 aa, Artificial Sequence
  Description of Artificial Sequence Synthetic peptide
SVCMA

SEQ ID NO: 53 - protein, 17 aa, Artificial Sequence
  Description of Artificial Sequence Synthetic peptide
VTREGLPQTGYADSVKG

SEQ ID NO: 54 - protein, 19 aa, Artificial Sequence
  Description of Artificial Sequence Synthetic peptide
RPTSPCTVDGELLASTYDY

SEQ ID NO: 55 - protein, 5 aa, Artificial Sequence
  Description of Artificial Sequence Synthetic peptide
SVCMA

SEQ ID NO: 56 - protein, 17 aa, Artificial Sequence
  Description of Artificial Sequence Synthetic peptide
VTREGLTKTGYADSVKG

SEQ ID NO: 57 - protein, 19 aa, Artificial Sequence
  Description of Artificial Sequence Synthetic peptide
RPTSPCTVDGELLASTYNY

SEQ ID NO: 58 - protein, 5 aa, Artificial Sequence
  Description of Artificial Sequence Synthetic peptide
SVCMA

SEQ ID NO: 59 - protein, 17 aa, Artificial Sequence
  Description of Artificial Sequence Synthetic peptide
VTREGLPQTGYADSVKG

SEQ ID NO: 60 - protein, 19 aa, Artificial Sequence
  Description of Artificial Sequence Synthetic peptide
RPTSPCTVDGELLASTYDY

SEQ ID NO: 61 - protein, 5 aa, Artificial Sequence
  Description of Artificial Sequence Synthetic peptide
SVCMA

SEQ ID NO: 62 - protein, 17 aa, Artificial Sequence
  Description of Artificial Sequence Synthetic peptide
VTREGLPQTGYADSVKG

SEQ ID NO: 63 - protein, 19 aa, Artificial Sequence
  Description of Artificial Sequence Synthetic peptide
RPTSPCTVDGELLASTYDY

SEQ ID NO: 64 - protein, 5 aa, Artificial Sequence
  Description of Artificial Sequence Synthetic peptide
SVCMA

SEQ ID NO: 65 - protein, 17 aa, Artificial Sequence
  Description of Artificial Sequence Synthetic peptide
VTREGLTKTGYADSVKG

SEQ ID NO: 66 - protein, 19 aa, Artificial Sequence
  Description of Artificial Sequence Synthetic peptide
RPTSPCTVDGELLASTYNY

SEQ ID NO: 67 - protein, 5 aa, Artificial Sequence
  Description of Artificial Sequence Synthetic peptide
SVCMA

SEQ ID NO: 68 - protein, 17 aa, Artificial Sequence
  Description of Artificial Sequence Synthetic peptide
VTREGLPQTGYADSVKG

SEQ ID NO: 69 - protein, 19 aa, Artificial Sequence
  Description of Artificial Sequence Synthetic peptide
RPTSPCTVDGELLASTYNY

SEQ ID NO: 70 - protein, 5 aa, Artificial Sequence
  Description of Artificial Sequence Synthetic peptide
SVCMA

SEQ ID NO: 71 - protein, 17 aa, Artificial Sequence
  Description of Artificial Sequence Synthetic peptide
VTREGLPQTGYADSVKG

SEQ ID NO: 72 - protein, 19 aa, Artificial Sequence
  Description of Artificial Sequence Synthetic peptide
RPTSPCTVDGELLASTYDY

SEQ ID NO: 73 - protein, 5 aa, Artificial Sequence
  Description of Artificial Sequence Synthetic peptide
SVCMA

SEQ ID NO: 74 - protein, 17 aa, Artificial Sequence
  Description of Artificial Sequence Synthetic peptide
VTREGLTKTGYADSVKG

SEQ ID NO: 75 - protein, 19 aa, Artificial Sequence
  Description of Artificial Sequence Synthetic peptide
RPTSPCTVDGELLASTYDY

SEQ ID NO: 76 - protein, 5 aa, Artificial Sequence
  Description of Artificial Sequence Synthetic peptide
SVCMA

SEQ ID NO: 77 - protein, 17 aa, Artificial Sequence
  Description of Artificial Sequence Synthetic peptide
VTREGLPQTGYADSVKG

SEQ ID NO: 78 - protein, 19 aa, Artificial Sequence
  Description of Artificial Sequence Synthetic peptide
RPTSPCTVDGELLASTYDY

SEQ ID NO: 79 - protein, 5 aa, Artificial Sequence
  Description of Artificial Sequence Synthetic peptide
SVCMA

SEQ ID NO: 80 - protein, 17 aa, Artificial Sequence
  Description of Artificial Sequence Synthetic peptide
VTREGLPQTGYADSVKG

SEQ ID NO: 81 - protein, 19 aa, Artificial Sequence
  Description of Artificial Sequence Synthetic peptide
RPTSPCTVDGELLASTYDY

SEQ ID NO: 82 - protein, 5 aa, Artificial Sequence
  Description of Artificial Sequence Synthetic peptide
SVCMA

SEQ ID NO: 83 - protein, 17 aa, Artificial Sequence
  Description of Artificial Sequence Synthetic peptide
VTREGLPQTGYADSVKG

SEQ ID NO: 84 - protein, 19 aa, Artificial Sequence
  Description of Artificial Sequence Synthetic peptide
RPTSPCTVDGELLASTYDY

SEQ ID NO: 85 - protein, 5 aa, Artificial Sequence
  Description of Artificial Sequence Synthetic peptide
SVCMA

SEQ ID NO: 86 - protein, 17 aa, Artificial Sequence
  Description of Artificial Sequence Synthetic peptide
VTREGLPQTGYADSVKG

SEQ ID NO: 87 - protein, 19 aa, Artificial Sequence
  Description of Artificial Sequence Synthetic peptide
RPTSPCTVDGELLASTYDY

SEQ ID NO: 88 - protein, 5 aa, Artificial Sequence
  Description of Artificial Sequence Synthetic peptide
SVCMA

SEQ ID NO: 89 - protein, 17 aa, Artificial Sequence
  Description of Artificial Sequence Synthetic peptide
VTREGLPQTGYADSVKG

SEQ ID NO: 90 - protein, 19 aa, Artificial Sequence
  Description of Artificial Sequence Synthetic peptide
RPTSPCTVDGELLASTYDY

SEQ ID NO: 91 - protein, 5 aa, Artificial Sequence
  Description of Artificial Sequence Synthetic peptide
SLCMA

SEQ ID NO: 92 - protein, 17 aa, Artificial Sequence
  Description of Artificial Sequence Synthetic peptide
VTRDGLRQTGYADSVKG

SEQ ID NO: 93 - protein, 19 aa, Artificial Sequence
  Description of Artificial Sequence Synthetic peptide
RPTSPCTVSGELIESDYSS

SEQ ID NO: 94 - protein, 5 aa, Artificial Sequence
  Description of Artificial Sequence Synthetic peptide
SLCMA

SEQ ID NO: 95 - protein, 17 aa, Artificial Sequence
  Description of Artificial Sequence Synthetic peptide
VTREGLPQTGYADSVKG

SEQ ID NO: 96 - protein, 19 aa, Artificial Sequence
  Description of Artificial Sequence Synthetic peptide
RPTSPCTVSGELIESDYSS

SEQ ID NO: 97 - protein, 5 aa, Artificial Sequence
  Description of Artificial Sequence Synthetic peptide
SLCMA

SEQ ID NO: 98 - protein, 17 aa, Artificial Sequence
  Description of Artificial Sequence Synthetic peptide
VTRDGLRQTGYADSVKG

SEQ ID NO: 99 - protein, 19 aa, Artificial Sequence
  Description of Artificial Sequence Synthetic peptide
RPTSPCTVSGELIESDYSS

SEQ ID NO: 100 - protein, 5 aa, Artificial Sequence
  Description of Artificial Sequence Synthetic peptide
SLCMA

SEQ ID NO: 101 - protein, 17 aa, Artificial Sequence
  Description of Artificial Sequence Synthetic peptide
VTRDGLRQTGYADSVKG

SEQ ID NO: 102 - protein, 19 aa, Artificial Sequence
  Description of Artificial Sequence Synthetic peptide
RPTSPCTVSGELIESDYSS

SEQ ID NO: 103 - protein, 140 aa, Artificial Sequence
  Description of Artificial Sequence Synthetic polypeptide
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICI